(19) United States
(12) Patent Application Publication
(10) Pub. No.: US 2025/0186431 A1
(43) Pub. Date: Jun. 12, 2025

(54) PHARMACEUTICAL COMPOSITION FOR RADIOTHERAPY, AND METHOD FOR TREATING SOLID CANCER USING SAME

(71) Applicant: Kyoto University, Kyoto (JP)

(72) Inventors: Fuyuhiko TAMANOI, Kyoto-shi, Kyoto (JP); Kotaro MATSUMOTO, Kyoto-shi, Kyoto (JP); Yuya HIGASHI, Kyoto-shi, Kyoto (JP)

(73) Assignee: Kyoto University, Kyoto (JP)

(21) Appl. No.: 18/846,521

(22) PCT Filed: Mar. 15, 2023
(86) PCT No.: PCT/JP2023/010023

§ 371 Date: Sep. 12, 2024

(30) Foreign Application Priority Data
Mar. 16, 2022 (JP) 2022-041624

(51) Int. Cl. A61K 31/496; A61K 9/14; A61K 41/00; A61K 47/02; A61P 35/00

(52) U.S. Cl. CPC A61K 31/496; A61K 9/143; A61K 41/00; A61K 47/02; A61P 35/00

(57) ABSTRACT

Provided is a pharmaceutical composition for use in radiotherapy, comprising: a compound represented by formula (I) wherein R¹ is an aryl group substituted with a substituent selected from an iodine atom and others, wherein the aryl group is optionally substituted with a substituent such as a hydroxy group, X represents a bond or the like and ring A is a group represented by formula (A), wherein R² is a non-aromatic heterocyclic group optionally substituted with at least one C₁-C₄ alkyl group, or a pharmaceutically acceptable salt thereof; and a pharmaceutically acceptable carrier. Also, provided is a nanoparticle and a method for treating solid cancer using the compound.

### TECHNICAL FIELD

The present invention relates to a pharmaceutical composition for radiotherapy, particularly X-ray therapy, and a method for treating solid cancer using the therapy. The present invention also relates to a nanoparticle that can be used for radiotherapy.

### BACKGROUND ART

As one of the radiotherapies currently under development, Auger therapy is known. The Auger therapy is a radiotherapy in which a high-Z atom (atom of high-Z element) such as gadolinium and iodine is localized within a cell and irradiated with X-rays. For example, a compound having an iodine atom is taken up by cancer cells and localized in the vicinity of DNA. Thereafter, when an X-ray having a predetermined energy is applied, the energy of X-ray is absorbed by the iodine atom to cause a photoelectric effect such as ejection of electrons of the K shell thereof. As a result, electrons such as Auger electrons having strong DNA cleaving and cancer-cell killing effects generate in the vicinity of DNA, producing a therapeutic effect. Auger electrons travel a short distance (hundreds of nanometers) but their DNA destructive effect and cytotoxic effect are high. For this reason, localization of a high-Z atom in the vicinity of DNA is important.

Up to the present, silica nanoparticles containing a high-Z atom suitable for use in Auger therapy have been proposed by a group of researchers including the present inventors (for example, Non Patent Literatures 1 and 2, Patent Literature 1).

Furthermore, Auger therapy using an iodine atom, for example, therapy using a nucleotide analogue, IUdR, is known (for example, Non Patent Literature 3). However, IUdR is rapidly metabolized in vivo and produces systemic toxicity. For this reason, it is difficult to use IUdR.

### CITATION LIST

### Patent Literature

Patent Literature 1: International Publication No. WO 2021/060498

### Non Patent Literature

Non Patent Literature 1: Scientific reports (2019), 9, 13275. Matsumoto et al. (https://doi.org/10.1038/s41598-019-49978-1)
Non Patent Literature 2: Scientific reports (2021), 11, 14192. Higashi et al. (https://doi.org/10.1038/s41598-021-93429-9)
Non Patent Literature 3: British Journal of Cancer (2004), 91, 544-551

### SUMMARY OF INVENTION

### Technical Problem

The present invention was made in consideration of the above circumstances. An object of the present invention is to provide a pharmaceutical composition and nanoparticle that can be used for radiotherapy, particularly, Auger therapy using X-rays and a method for treating solid cancer using the same.

### Solution to Problem

The present invention includes the following embodiments but the invention is not limited to them.

[1]A pharmaceutical composition (hereinafter also referred to as “the pharmaceutical composition of the present invention”) for use in radiotherapy, comprising: a compound represented by formula (I):

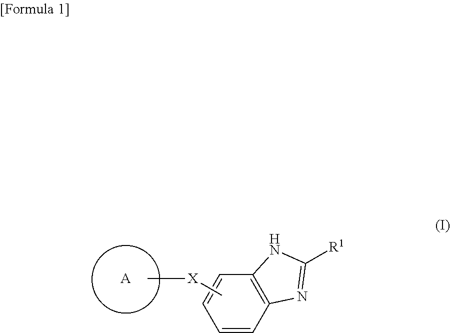

wherein
   R¹ is an aryl group substituted with at least one substituent selected from the group consisting of an iodine atom, a C₁-C₄ alkyl group substituted with at least one iodine atom, an aryl group substituted with at least one iodine atom, a gadolinium atom-containing group, a gold atom-containing group, a silver atom-containing group and a platinum atom-containing group, wherein the aryl group is optionally substituted with at least one substituent selected from the group consisting of a hydroxy group, a C₁-C₄ alkyl group and a C₁-C₄ alkoxy group,
   X represents a bond or a C₂ or C₄ alkenylene group, and
   ring A is a group represented by formula (A):

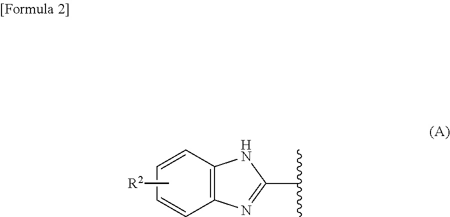

wherein
   R² is a non-aromatic heterocyclic group containing one or more heteroatoms selected from the group consisting of a nitrogen atom, an oxygen atom and a sulfur atom, wherein at least one of the heteroatoms is a nitrogen atom, the non-aromatic heterocyclic group optionally substituted with at least one C₁-C₄ alkyl group, and

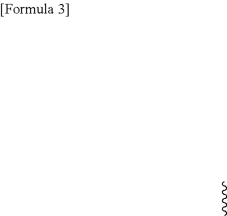

represents the attachment to the group X, or a pharmaceutically acceptable salt thereof (hereinafter also referred to as “the compound of the present invention); and a pharmaceutically acceptable carrier.

[2] The pharmaceutical composition according to [1], wherein R¹ is an aryl group substituted with at least one iodine atom, wherein the aryl group is optionally substituted with at least one substituent selected from the group consisting of a hydroxy group, a C₁-C₄ alkyl group and a C₁-C₄ alkoxy group, and
R² is a 6-membered non-aromatic heterocyclic group containing 1 to 4 nitrogen atoms, optionally substituted with at least one C₁-C₄ alkyl group.

[3] The pharmaceutical composition according to [1] or [2], wherein the compound represented by formula (I) is a compound represented by formula (II):

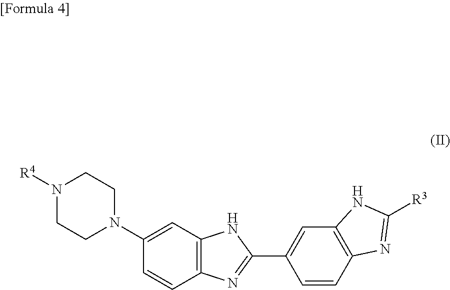

wherein
   R³ is a phenyl group substituted with 1 to 3 iodine atoms, wherein the phenyl group is optionally substituted with 1 or 2 hydroxy groups, and
   R⁴ is a hydrogen atom or a C₁-C₄ alkyl group.

[4] The pharmaceutical composition according to any one of [1] to [3], wherein the radiation is an X-ray.

[5] The pharmaceutical composition according to [4], wherein the X-ray is an X-ray that can excite K shell electrons of an atom selected from an iodine atom, a gadolinium atom, a gold atom, a silver atom and a platinum atom of the compound represented by formula (I).

[6] The pharmaceutical composition according to [4] or [5], wherein the X-ray is a monochromatic X-ray or a characteristic X-ray.

[7] The pharmaceutical composition according to any one of [1] to [6], for treating solid cancer or suppressing enlargement or growth of solid cancer.

[8] The pharmaceutical composition according to [7], wherein the solid cancer is a brain tumor, lung cancer, ovarian cancer, digestive system cancer, osteosarcoma or head and neck cancer.

[9] The pharmaceutical composition according to [7] or [8], wherein the solid cancer is in a hypoxic condition.

[10]A nanoparticle (hereinafter also referred to as “the nanoparticle of the present invention”) comprising:
a compound represented by formula (I):

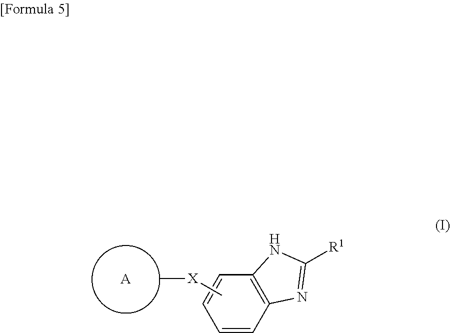

wherein
R¹ is an aryl group substituted with at least one substituent selected from the group consisting of an iodine atom, a C₁-C₄ alkyl group substituted with at least one iodine atom, an aryl group substituted with at least one iodine atom, a gadolinium atom-containing group, a gold atom-containing group, a silver atom-containing group and a platinum atom-containing group, wherein the aryl group is optionally substituted with at least one substituent selected from the group consisting of a hydroxy group, a C₁-C₄ alkyl group and a C₁-C₄ alkoxy group,
X represents a bond or a C₂ or C₄ alkenylene group, and
ring A is a group represented by formula (A):

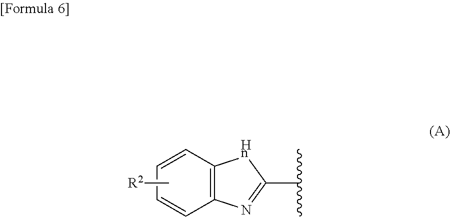

wherein
R² is a non-aromatic heterocyclic group containing one or more heteroatoms selected from the group consisting of a nitrogen atom, an oxygen atom and a sulfur atom, wherein at least one of the heteroatoms is a nitrogen atom, the non-aromatic heterocyclic group optionally substituted with at least one C₁-C₄ alkyl group, and

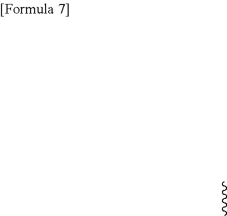

represents the attachment to the group X,
or a pharmaceutically acceptable salt thereof; and
a porous silica carrier.

[10-1]A nanoparticle of porous silica carrying an iodine atom-containing Hoechst compound (IH: Iodine-Hoechst).

[11] The nanoparticle according to [10], wherein the nanoparticle is a biodegradable mesoporous silica nanoparticle.

[11-1] The nanoparticle according to [10] or [11] for X-ray irradiation.

[12]A method for treating solid cancer or suppressing enlargement or growth of solid cancer, comprising irradiating, with an X-ray, the compound represented by formula (I) or a pharmaceutically acceptable salt thereof according to [1], or the pharmaceutical composition according to any one of [1] to [9], or the nanoparticle according to [10] or [11], taken in a body of a subject, to destroy a cancer cell.

[13] The pharmaceutical composition according to any one of [4] to [9], nanoparticle according to [11-1] or method according to [12], wherein the compound represented by formula (I) contains an iodine atom and the X-ray is a monochromatic X-ray having an energy of 33.2 keV.

[14] The compound represented by formula (I) or a pharmaceutically acceptable salt thereof according to [1], pharmaceutical composition according to any one of [1] to [9] or nanoparticle according to [10], [11] or [11-1] for use in treating solid cancer or suppressing enlargement or growth of solid cancer.

[15] Use of the compound represented by formula (I) or a pharmaceutically acceptable salt thereof according to [1], pharmaceutical composition according to any one of [1] to [9] or nanoparticle according to [10], [11] or [11-1] for producing a medical drug for treating solid cancer or suppressing enlargement or growth of solid cancer.

### Advantageous Effect of Invention

According to the present invention, it is possible to perform highly effective radiotherapy using the Auger effect.

### BRIEF DESCRIPTION OF DRAWINGS

FIG. 1 shows the results (images and calculation values) of docking simulation of an iodine atom-containing Hoechst compounds (4 types) and DNA, viewed along the axis of the helix.

FIG. 2 shows the results (images) of docking simulation of an iodine atom-containing Hoechst compounds (4 types) and DNA, viewed perpendicular to the axis of the helix.

FIG. 3 shows the images of microscopic observation on cancer cell OVCAR8. FIG. 3A shows cancer cell OVCAR8 emitting a fluorescence (green).

FIG. 3B shows a region stained in blue with a Hoechst compound serving as a dye. FIG. 3C shows a processed image obtained by superimposing FIG. 3A and FIG. 3B.

FIG. 4 shows results (images) of microscopic observation on tumor spheroids.

FIG. 5 schematically shows the setup of a monochromatic X-ray irradiation device.

FIG. 6 schematically shows a rack of tumor spheroid samples in the monochromatic X-ray irradiation device.

FIG. 7 shows the results (images) of microscopic observation on tumor spheroids after irradiation with monochromatic X-rays (followed by incubation).

FIG. 8 shows the results (images) of microscopic observation on tumor spheroids after irradiation with monochromatic X-rays (immediately after the irradiation).

FIG. 9 shows the results (graph) of a cytotoxicity test of iodine atom-containing Hoechst compounds.

FIG. 10 shows the results (images) of microscopic observation on tumor spheroids in a test performed in a hypoxic condition.

FIG. 11 shows a graph indicating changes over time of the fluorescence signal intensity of a hypoxic marker HIF-1α.

FIG. 12 shows the results of tumor spheroids (images at the bottom of a tube) irradiated with X-rays in the test performed in a hypoxic condition.

FIG. 13 shows a TEM image of a mesoporous silica nanoparticle.

FIG. 14 shows absorption spectra of a solution and washing solution used for producing nanoparticles, measured by a spectrophotometer.

FIG. 15 shows the results of nuclei stained with IH-BPMO.

FIG. 16 shows the results of a cancer spheroid destruction experiment using IH-BPMO.

### DESCRIPTION OF EMBODIMENTS

### Definition

First of all, terms will be defined to explain the present invention.

As used herein, an alkyl group refers to an aliphatic saturated hydrocarbon group constituted of carbon atoms and hydrogen atoms. The alkyl group may be linear or branched. A C₁-C₄ alkyl group represents an alkyl group having 1 to 4 carbon atoms. Examples of the C₁-C₄ alkyl group include, but are not limited to, a methyl group, an ethyl group, a n-propyl group, an isopropyl group, a n-butyl group, an isobutyl group, a sec-butyl group and a tert-butyl group.

As used herein, an alkoxy group refers to a group represented by an (alkyl)-O—. The meaning of the alkyl is as defined above. A C₁-C₄ alkoxy group refers to an alkoxy group having 1 to 4 carbon atoms. Examples of the C₁-C₄ alkoxy group include, but are not limited to, a methoxy group, an ethoxy group, a n-propoxy group, an isopropoxy group, a n-butoxy group and a tert-butoxy group.

As used herein, an aryl group refers to an aromatic hydrocarbon group. An aryl group preferably has 6 to 12 carbon atoms, more preferably 6 to 10 carbon atoms, and further preferably 6 to 8 carbon atoms.

Examples of the aryl group include, but are not limited to, a phenyl group, a naphthyl group, a phenanthryl group and an anthracenyl group.

As used herein, an alkenylene group refers to a divalent unsaturated hydrocarbon group having at least one carbon-carbon double bond. A C₂ or C₄ alkenylene group refers to an alkenylene group having 2 or 4 carbon atoms, and specific examples thereof include —C═C— and, —C═C—C═C—.

As used herein, a non-aromatic heterocyclic group refers to a non-aromatic cyclic group having a ring, which is constituted of at least one hetero atom selected from the group consisting of a nitrogen atom, an oxygen atom and a sulfur atom and the rest of carbon atoms. Note that, in the non-aromatic heterocyclic group in a compound represented by formula (I), at least one of the heteroatoms is a nitrogen atom. The non-aromatic heterocyclic group has preferably a 4 to 8 membered ring, more preferably a 5 membered ring or 6 membered ring, and further preferably a 6 membered ring. The non-aromatic heterocyclic group may have a double bond in the ring as long as it maintains a non-aromatic system or may not have a double bond in the ring. The non-aromatic heterocyclic group may be a saturated or unsaturated cyclic group but is more preferably a saturated cyclic group. Examples of the non-aromatic heterocyclic group include, but are not limited to, a piperidinyl group, a piperazinyl group, a pyrrolidinyl group, an imidazolidinyl group, a pyrazolidinyl group, a morpholinyl group, a thiomorpholinyl group, an oxazolidinyl group and a thiazolidinyl group.

As used herein, the “bond” in the definition of group X means that two moieties linked to X are directly bound in the chemical structural formula (more specifically, X is not substantially present). More specifically, for example, the structure represented by ring A-X-benzene ring (see, formula (I)), if X represents a bond, represents ring A-benzene ring (more specifically, ring A and a benzene ring are directly bound).

As used herein, a hydroxy group refers to a group represented by —OH.

### Compound

The pharmaceutical composition of the present invention comprises a compound represented by formula (I) or a pharmaceutically acceptable salt thereof:

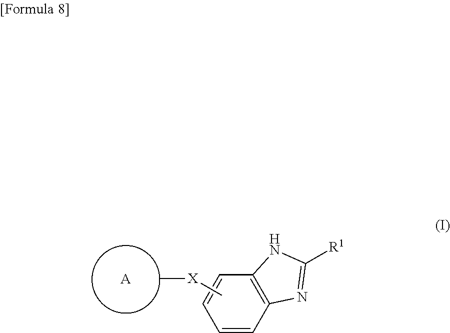

In the formula (I),
R¹ is an aryl group substituted with at least one substituent selected from the group consisting of an iodine atom, a C₁-C₄ alkyl group substituted with at least one iodine atom, an aryl group substituted with at least one iodine atom, a gadolinium atom-containing group, a gold atom-containing group, a silver atom-containing group and a platinum atom-containing group, wherein the aryl group is optionally substituted with at least one substituent selected from the group consisting of a hydroxy group, a C₁-C₄ alkyl group and a C₁-C₄ alkoxy group,
X represents a bond or a C₂ or C₄ alkenylene group; and
ring A is a group represented by formula (A):

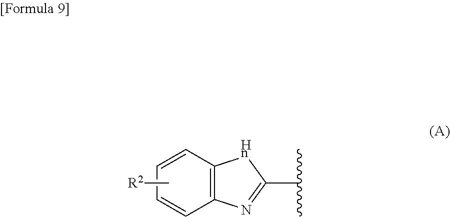

wherein
R² is a non-aromatic heterocyclic group containing one or more heteroatoms selected from the group consisting of a nitrogen atom, an oxygen atom and a sulfur atom, wherein at least one of the heteroatoms is a nitrogen atom, the non-aromatic heterocyclic group optionally substituted with at least one C₁-C₄ alkyl group, and

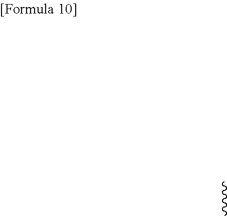

(more specifically, wave line) represents the attachment to the group X.

A compound represented by formula (I) has a chemical structure, in brief, in which group R¹ is bound to the imidazolyl group of a benzimidazole ring structure positioned on the right side, and ring A (another benzimidazole ring structure positioned on the left side) is bound to the phenyl group of the right-side benzimidazole ring structure via group X.

R¹ can be an aryl group substituted with at least one substituent selected from the group consisting of an iodine atom, a C₁-C₄ alkyl group substituted with at least one iodine atom, a gadolinium atom-containing group, a gold atom-containing group, a silver atom-containing group and a platinum atom-containing group. In the compound of the present invention, R¹ has an aryl group as a basic skeleton and the aryl group binds to a benzimidazole ring structure. The aryl group serving as the basic skeleton of R¹ is as defined above and preferably a phenyl group or a naphthyl group but not limited to these.

R¹ is preferably an aryl group substituted with at least one substituent selected from the group consisting of an iodine atom, a C₁-C₄ alkyl group substituted with at least one iodine atom and an aryl group substituted with at least one iodine atom. More specifically, in a preferred embodiment, R¹ contains an iodine atom (I atom). The substituent for an aryl group represented by R¹ can be at least one iodine atom. It is satisfactory if the number of the iodine atom serving as a substituent is preferably one or more. The number of iodine atoms may be, for example, 1 to 10, preferably 1 to 7, and more preferably 1 to 5. The substituent for an aryl group represented by R¹ may be a C₁-C₄ alkyl group substituted with at least one iodine atom. The C₁-C₄ alkyl group that may serve as a substituent is as defined above and is preferably, a methyl group or an ethyl group (wherein the C₁-C₄ alkyl group that may serve as a substituent for an aryl group represented by R¹ is substituted with at least one iodine atom). Examples of such a substituent include an iodomethyl group and an iodoethyl group. Furthermore, the substituent for an aryl group represented by R¹ may be an aryl group substituted with at least one iodine atom. The aryl group that may serve as a substituent is as defined above. Examples of the aryl group preferably include, but are not limited to, a phenyl group and a naphthyl group (wherein the aryl group which may serve as a substituent for an aryl group represented by R¹ is substituted with at least one iodine atom). Examples of such a substituent may include an iodophenyl group. Of these, R¹ more preferably represents an aryl group substituted with at least one iodine atom (more specifically, a substituent for an aryl group represented by R¹ is at least one iodine atom).

Furthermore, in another aspect, R¹ may be an aryl group substituted with at least one substituent selected from the group consisting of a gadolinium atom-containing group, a gold atom-containing group, a silver atom-containing group and a platinum atom-containing group. The gadolinium atom-containing group refers to a group containing a gadolinium atom (Gd) itself or containing a gadolinium atom and appropriate number and types of ligands (for example, a monodentate ligand such as a halogen atom (for example, chlorine atom, fluorine atom) and a phosphine (for example, triphenylphosphine), a bidentate ligand such as acetonate (for example, acetylacetonate) and a multidentate ligand such as ethylenediaminetetraacetic acid). The gadolinium atom-containing group refers to a group containing a gadolinium atom (Gd) and may be a group binding to an aryl group represented by R¹ directly by the gadolinium atom or via a ligand to which the gadolinium atom is bound. For example, in the gadolinium atom-containing group, the gadolinium atom may be present as a part of a complex structure of gadopentetic acid (Gd (III) DTPA) and the DTPA moiety of the gadopentetic acid may bind to an aryl group represented by R¹. DTPA herein refers to diethylenetriaminepenta acetic acid. In gadopentetic acid, Gd³⁺ and a DTPA ion form into a complex. Because of this, the acid of DTPA, which serves as a reactive group, may bind to an aryl group. Needless to say, the gadolinium atom-containing group is not limited to the above aspect.

The gold atom-containing group refers to a group containing a gold atom (Au) itself or containing a gold atom and an appropriate number and types of ligands (for example, a monodentate ligand such as a halogen atom (for example, chlorine atom, fluorine atom) and a phosphine (for example, triphenylphosphine), a bidentate ligand such as acetonate (for example, acetylacetonate) and a multidentate ligand such as ethylenediaminetetraacetic acid). The gold atom-containing group refers to a group containing a gold atom (Au) and binding to an aryl group represented by R¹ directly by a gold atom or via a ligand to which the gold atom is bound. Furthermore, a silver atom-containing group refers to a group containing a silver atom (Ag) itself or containing a silver atom and an appropriate number and types of ligands (for example, a monodentate ligand such as a halogen atom (for example, chlorine atom, fluorine atom) and a phosphine (for example, triphenylphosphine), a bidentate ligand such as acetonate (for example, acetylacetonate) and a multidentate ligand such as ethylenediaminetetraacetic acid). The silver atom-containing group refers to a group containing a silver atom (Ag) and binding to an aryl group represented by R¹ directly by the silver atom or via a ligand to which the silver atom is bound. The platinum atom-containing group refers to a group containing a platinum atom (Pt) itself or containing a platinum atom and an appropriate number and types of ligands (for example, a monodentate ligand such as a halogen atom (for example, chlorine atom, fluorine atom) and a phosphine (for example, triphenylphosphine), a bidentate ligand such as acetonate (for example, acetylacetonate) and a multidentate ligand such as ethylenediaminetetraacetic acid). The platinum atom-containing group refers to a group containing a platinum atom (Pt) and binding to an aryl group represented by R¹ directly by the platinum atom or via a ligand to which the platinum atom is bound.

A group containing each of these metal atoms (gold, silver or platinum atom) may bind to at least one oxygen atom or a ligand containing an oxygen atom through the metal atom and then binds to an aryl group represented by R¹ via the oxygen atom. Needless to say, the metal atom (gold, silver or platinum atom) containing group is not limited to the above aspect.

Examples of the substitution in an aryl group represented by R¹ may include a substitution of a single-site with a single type of substituent, substations of a plural of sites with a single type of substituent and substations of a plural of sites with a plurality of types of substituents. For example, a substituent may be constituted of both an iodine atom and a C₁-C₄ alkyl group substituted with at least one iodine atom. Alternatively, a substituent may be constituted of both an iodine atom and a gadolinium atom-containing group. Alternatively, for example, a substituent may be constituted of 2 or more, 3 or more, or 4 or more iodine atoms.

As mentioned above, the compound of the present invention inevitably contains at least one atom selected from the group consisting of an iodine atom, a gadolinium atom, a gold atom, a silver atom and a platinum atom as an atom serving as an X-ray irradiation target (hereinafter, sometimes referred to as “X-ray irradiation target atom”) in a substituent for an aryl group represented by R¹. These atoms are those capable of easily ejecting Auger electrons by X-ray irradiation, as described later (referred also as high-Z atoms). Accordingly, if these atoms are present, it is possible to perform effective radiotherapy using the Auger effect.

The aryl group represented by R¹ and serving as a basic skeleton may further be optionally substituted with at least one substituent selected from the group consisting of a hydroxy group, a C₁-C₄ alkyl group and a C₁-C₄ alkoxy group. If such a substituent is present, it is possible to obtain, e.g., an effect of changing the properties (for example, stability and safety) of the compound itself and an effect of controlling the binding to another compound (for example, enhancing binding affinity between the compound and DNA). The hydroxy group, C₁-C₄ alkyl group and C₁-C₄ alkoxy group are as defined above. The substituent is more preferably a hydroxy group. If so, for example, a binding affinity can be enhanced.

Ring A has a benzimidazole ring structure. To the imidazolyl group of the benzimidazole ring structure, group X is bound, at the same time, group R² is bound to the phenyl group of the benzimidazole ring structure.

R² has, as a basic skeleton, a non-aromatic heterocyclic group containing one or more heteroatoms selected from the group consisting of a nitrogen atom, an oxygen atom and a sulfur atom, wherein at least one of the heteroatoms is a nitrogen atom. The non-aromatic heterocyclic group is optionally substituted with at least one C₁-C₄ alkyl group. The non-aromatic heterocyclic group may be an unsubstituted one.

Preferably, the non-aromatic heterocyclic group is substituted with at least one C₁-C₄ alkyl group. R² may be a 6-membered non-aromatic heterocyclic group containing 1 to 4 nitrogen atoms optionally substituted with at least one C₁-C₄ alkyl group. The non-aromatic heterocyclic group is as defined above. Examples of the non-aromatic heterocyclic group may include, but are not limited to, a piperidinyl group, a piperazinyl group, a pyrrolidinyl group, an imidazolidinyl group, a pyrazolidinyl group, a morpholinyl group, a thiomorpholinyl group, an oxazolidinyl group and a thiazolidinyl group. The non-aromatic heterocyclic group is, preferably, a piperidinyl group, a piperazinyl group, a pyrrolidinyl group, an imidazolidinyl group and a pyrazolidinyl group, and more preferably, a piperidinyl group and a piperazinyl group. A nitrogen atom of the non-aromatic heterocyclic group may bind to the phenyl group of ring A or a carbon atom thereof may bind to the phenyl group of ring A. The C₁-C₄ alkyl group that may serve as a substituent is as defined above and examples thereof include a methyl group and an ethyl group. If the C₁-C₄ alkyl group is present, it may bind to a nitrogen atom or a carbon atom of a non-aromatic heterocyclic group. When R² binds to the phenyl group of ring A, the position on the phenyl group is not particularly limited but may be the position of a carbon atom next to the carbon atom shared by an imidazole ring structure of ring A and a benzene ring structure or further the carbon atom next to the carbon atom (the carbon atom next to the carbon atom shared by an imidazole ring structure and a benzene ring structure). The latter one is more preferable.

X represents a bond or a C₂ alkenylene group or a C₄ alkenylene group. When X represents a bond, ring A directly binds to the phenyl group of a benzimidazole ring structure. In this case, a conjugated electron system is formed between the two benzimidazole ring structures. Due to the conjugated electron system, the compound may have a property such as binding affinity for DNA. Furthermore, if X is an alkenylene group, ring A binds to the phenyl group of the benzimidazole ring structure via the alkenylene group. If the cases where X is a C₂ alkenylene group (—C═C—) and a C₄ alkenylene group (—C═C—C═C—), a conjugated electron system is also formed between the two benzimidazole ring structures via the alkenylene group. Due to the conjugated electron system, the compound may have a property such as binding affinity for DNA. X is more preferably a bond. When X binds to the phenyl group of a benzimidazole ring structure represented by formula (I), the position on the phenyl group is not particularly limited but may be a carbon atom next to the carbon atom shared with an imidazole ring structure and a benzene ring structure and further the carbon atom next to the carbon atom (the carbon atom next to the carbon atom shared with an imidazole ring structure and a benzene ring structure). The latter one is more preferable.

In a preferred embodiment, R¹ is an aryl group substituted with at least one iodine atom. The aryl group herein is optionally substituted with at least one substituent selected from the group consisting of a hydroxy group, a C₁-C₄ alkyl group and a C₁-C₄ alkoxy group. R² herein is a 6-membered non-aromatic heterocyclic group containing 1 to 4 nitrogen atoms optionally substituted with at least one C₁-C₄ alkyl group. Further, X is more preferably a bond.

Herein, in R¹ which is an aryl group substituted with at least one iodine atom, a maximum number of iodine atoms can be determined depending on the number of other optional substituents (the number of bonds of aryl groups). If the aryl group is a phenyl group, since the phenyl group may have 5 available bonds for a substituent excluding the bond to a benzimidazole ring structure, the number of iodine atoms can be 1, 2, 3, 4 or 5 (maximum). However, in this case, the number of optional substituents is limited to 4 (maximum) or less, 3 or less, 2 or less, 1 or less, 0 (the total number of iodine atoms and optional substituents becomes 5 or less). The larger the number of iodine atoms is, the higher the Auger effect can be obtained.

In a preferred embodiment, a compound represented by formula (I) is represented by formula (II):

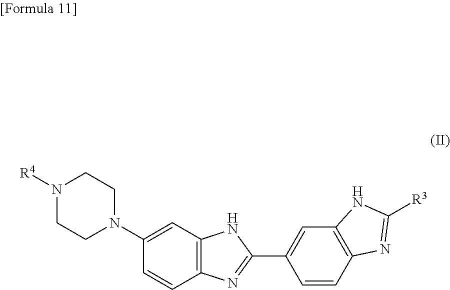

wherein R³ is a phenyl group substituted with 1 to 3 iodine atoms, wherein the phenyl group is optionally substituted with 1 or 2 hydroxy groups, and R⁴ is a hydrogen atom or a C₁-C₄ alkyl group.

Compared to the structure of formula (I), the structure of formula (II) has a chemical structure, in brief, in which X in formula (I) is a bond, the binding position of two benzimidazole ring structures is specified, group R¹ is replaced with more limited group R³, and group R² is replaced with a piperazinyl group having group R⁴ as a substituent.

R³ is preferably a phenyl group substituted with one or two iodine atoms that may further be optionally substituted with a single hydroxy group and more preferably a phenyl group substituted with a single iodine atom that may further be optionally substituted with a single hydroxy group. Alternatively, R³ may be a phenyl group substituted only with 1 or 2 iodine atoms.

R⁴ is preferably a C₁-C₄ alkyl group and further more preferably a methyl group or an ethyl group.

The compounds represented by formula (I) and formula (II) each can be used in the form as it is (free form, not a salt). Furthermore, the compounds represented by formula (I) and formula (II) may be used in a pharmaceutical composition in the form of a pharmaceutically acceptable salt thereof. As used herein, “pharmaceutically acceptable” means that the substance is non-toxic to a subject when applied (for example, administered) to the subject. The salt may be an acid addition salt or a base addition salt but an acid addition salt is preferable. This is apparent from the content of a plurality of nitrogen atoms, as shown in formula (I) and formula (II). Examples of the acid addition salt include inorganic salts such as a hydrochloride, a sulfate and a nitrate; and organic salts such as an acetate, a sulfonate and a citrate. Note that, examples of the acid addition salt include a salt of a plurality of acid molecules (for example, a salt of a plurality of hydrochloric acid molecules, more specifically dihydrochloride and trihydrochloride).

The compounds represented by formula (I) and formula (II) may be compounds (isotope-labeled compounds) having atoms partly substituted with an isotope (for example, deuterium (²H), carbon 13 (¹³C), nitrogen 15 (¹⁵N)). The compounds labeled with such an isotope are included in the compound of the present invention.

Furthermore, if the compounds represented by formula (I) and formula (II) have isomers (for example, stereoisomers), the isomers (for example, stereoisomers, for example, racemates and enantiomers) and a mixture of the isomers are included in the compound of the present invention.

Particularly preferred embodiments of the compound of the present invention will be described below.

### Embodiment 1

A compound represented by formula (I), wherein R¹ is an aryl group substituted with at least one substituent selected from the group consisting of an iodine atom and a C₁-C₄ alkyl group substituted with at least one iodine atom, wherein the aryl group is optionally substituted with at least one substituent selected from the group consisting of a hydroxy group, a C₁-C₄ alkyl group and a C₁-C₄ alkoxy group; X represents a bond; and R² is a 6-membered non-aromatic heterocyclic group containing 1 to 4 nitrogen atoms, optionally substituted with at least one C₁-C₄ alkyl group.

### Embodiment 2

A compound represented by formula (I), wherein R¹ is an aryl group substituted with at least one iodine atom, wherein the aryl group is optionally substituted with at least one substituent selected from the group consisting of a hydroxy group, a C₁-C₄ alkyl group and a C₁-C₄ alkoxy group; X represents a bond; and R² is a 6-membered non-aromatic heterocyclic group containing 1 to 4 nitrogen atoms, optionally substituted with at least one C₁-C₄ alkyl group.

### Embodiment 3

A compound represented by formula (I), wherein R¹ is a phenyl group substituted with at least one substituent selected from the group consisting of an iodine atom and a C₁-C₄ alkyl group substituted with at least one iodine atom, wherein the phenyl group is optionally substituted with at least one substituent selected from the group consisting of a hydroxy group, a C₁-C₄ alkyl group and a C₁-C₄ alkoxy group; X represents a bond; and R² is a 6-membered non-aromatic heterocyclic group containing 1 to 4 nitrogen atoms, optionally substituted with at least one C₁-C₄ alkyl group.

### Embodiment 4

A compound represented by formula (I), wherein R¹ is a phenyl group substituted with at least one iodine atom, wherein the phenyl group is optionally substituted with at least one substituent selected from the group consisting of a hydroxy group, a C₁-C₄ alkyl group and a C₁-C₄ alkoxy group; X represents a bond; and R² is a 6-membered non-aromatic heterocyclic group containing 1 to 4 nitrogen atoms, optionally substituted with at least one C₁-C₄ alkyl group.

### Embodiment 5

A compound represented by formula (I), wherein R¹ is a phenyl group substituted with at least one iodine atom, wherein the phenyl group is optionally substituted with at least one substituent selected from the group consisting of a hydroxy group, a C₁-C₄ alkyl group and a C₁-C₄ alkoxy group; X represents a bond; and R² is a piperidinyl group or piperazinyl group optionally substituted with at least one C₁-C₄ alkyl group.

### Embodiment 6

A compound represented by formula (I), wherein R¹ is a phenyl group substituted with at least one iodine atom, wherein the phenyl group is optionally substituted with at least one hydroxy group; X represents a bond; and R² is a piperazinyl group optionally substituted with at least one C₁-C₄ alkyl group.

### Embodiment 7

A compound represented by formula (I), wherein R¹ is a phenyl group substituted with at least one iodine atom, wherein the phenyl group is optionally substituted with at least one hydroxy group; X represents a bond; and R² is a piperazinyl group optionally substituted with at least one methyl group.

### Embodiment 8

A compound represented by formula (I), wherein R¹ is a phenyl group substituted with at least one iodine atom, wherein the phenyl group is optionally substituted with at least one hydroxy group; X represents a bond; and R² a piperazinyl group optionally substituted with a methyl group.

### Embodiment 9

A compound represented by formula (I), wherein R¹ is a phenyl group substituted with 1 to 3 iodine atoms, wherein the phenyl group is optionally substituted with one or two hydroxy groups; X represents a bond; and R² is a piperazinyl group optionally substituted with at least one methyl group.

### Embodiment 10

A compound represented by formula (I), wherein R¹ is a phenyl group substituted with 1 to 3 iodine atoms, wherein the phenyl group is optionally substituted with one or two hydroxy groups; X represents a bond; and R² is a piperazinyl group substituted with a methyl group.

### Embodiment 11

A compound represented by formula (I), wherein R¹ is a phenyl group substituted with at least one C₁-C₄ alkyl group substituted with at least one iodine atom, wherein the phenyl group is optionally substituted with at least one hydroxy group; X represents a bond; and R² is a piperazinyl group optionally substituted with at least one C₁-C₄ alkyl group.

### Embodiment 12

A compound represented by formula (I), wherein R¹ is a phenyl group substituted with at least one substituent selected from the group consisting of an iodine atom and a C₁-C₄ alkyl group substituted with at least one iodine atom, wherein the phenyl group is optionally substituted with at least one substituent selected from the group consisting of a hydroxy group, a C₁-C₄ alkyl group and a C₁-C₄ alkoxy group; X is a C₂ or C₄ alkenylene group; and R² is a 6-membered non-aromatic heterocyclic group containing 1 to 4 nitrogen atoms, optionally substituted with at least one C₁-C₄ alkyl group.

### Embodiment 13

A compound represented by formula (I), wherein R¹ is a phenyl group substituted with at least one iodine atom, wherein the phenyl group is optionally substituted with at least one hydroxy group; X is a C₂ or C₄ alkenylene group; and R² is a piperazinyl group optionally substituted with at least one C₁-C₄ alkyl group.

### Embodiment 14

A compound represented by formula (II), wherein R³ is a phenyl group substituted with 1 to 3 iodine atoms, wherein the phenyl group is optionally substituted with 1 or 2 hydroxy groups; and R⁴ is a hydrogen atom, a methyl group, an ethyl group, a propyl group or an isopropyl group.

### Embodiment 15

A compound represented by formula (II), wherein R³ is a phenyl group substituted with 1 to 3 iodine atoms, wherein the phenyl group is optionally substituted with a single hydroxy group; and R⁴ is a hydrogen atom, a methyl group or an ethyl group.

### Embodiment 16

A compound represented by formula (II), wherein R³ is a phenyl group substituted with 1 to 3 iodine atoms, wherein the phenyl group is optionally substituted with a single hydroxy group; and R⁴ is a methyl group.

### Embodiment 17

A compound represented by formula (II), wherein R³ is a phenyl group substituted with 1 or 2 iodine atoms, wherein the phenyl group is optionally substituted with a single hydroxy group; and R⁴ is a methyl group.

### Embodiment 18

A compound represented by formula (II), wherein R³ is a phenyl group substituted with a single iodine atom, wherein the phenyl group is optionally substituted with a single hydroxy group; and R⁴ is a methyl group.

### Embodiment 19

A compound represented by formula (II), wherein R³ is a phenyl group substituted with 1 to 3 iodine atoms; and R⁴ is a methyl group.

### Embodiment 20

A compound represented by formula (II), wherein R³ is a phenyl group substituted with a single iodine atom; and R⁴ is a methyl group.

### Embodiment 21

A compound represented by formula (II), wherein R³ is a phenyl group substituted with 1 to 3 iodine atoms and a single hydroxy group; and R⁴ is a methyl group.

### Embodiment 22

A compound represented by formula (II), wherein R³ is a phenyl group substituted with a single iodine atom and a single hydroxy group; and R⁴ is a methyl group.

Examples of the compounds represented by formula (I) and formula (II) will be more specifically shown below.

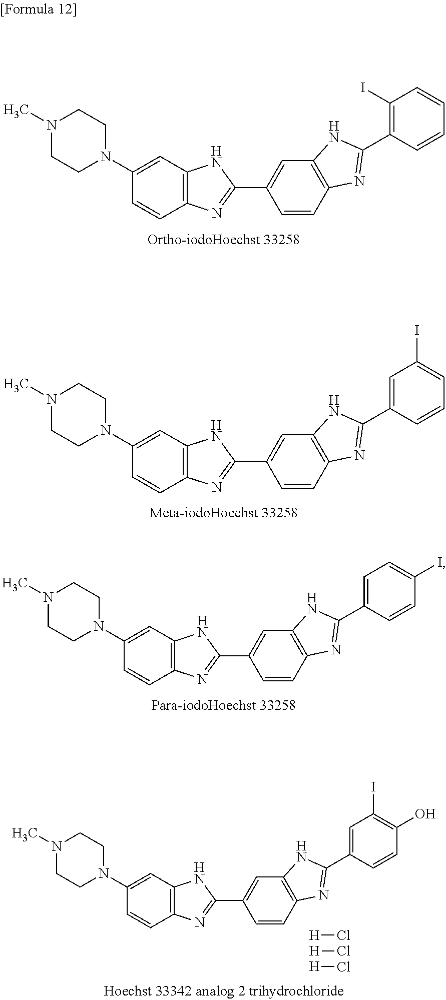

The above compounds are commonly known and commercially available. The compounds are commercially available under the name of “Ortho-iodoHoechst 33258” (MW=534.4), “Meta-iodoHoechst 33258” (MW=534.4), “Para-iodoHoechst 33258” (MW=534.4) and “Hoechst 33342 analog 2 trihydrochloride” (MW=659.8) (MW stands for molecular weight). These compounds are derivatives or analogs of the following Hoechst compounds (compounds used for Hoechst stain) commonly used.

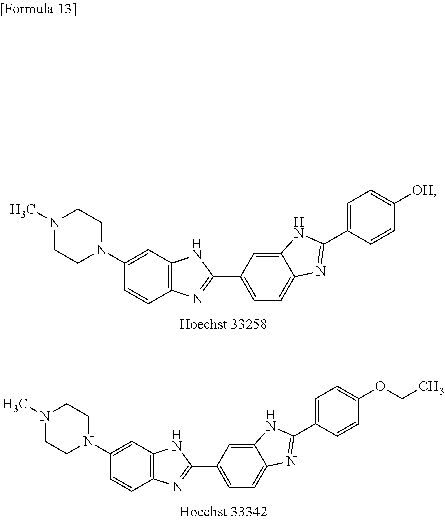 [SMILES: CN1CCN(c2ccc3nc(-c4ccc5nc(-c6ccc(O)cc6)[nH]c5c4)[nH]c3c2)CC1 ; CCOc1ccc(-c2nc3ccc(-c4nc5ccc(N6CCN(C)CC6)cc5[nH]4)cc3[nH]2)cc1]

Hoechst stains are commonly known as a method for staining DNA in a cell. Hoechst dyes (Hoechst compounds) have a high binding affinity for DNA and the DNA stained with a Hoechst stain emits a blue fluorescence. In a particularly preferred aspect (above), the compound of the present invention has almost the same structure as that of a common Hoechst compound except that an iodine atom is introduced. In this sense, the compound of the present invention can be referred to as “iodine atom-containing Hoechst compound” (may be called as an iodized Hoechst compound). In addition, the iodine atom-containing Hoechst compound has a high binding affinity for DNA similarly to a common Hoechst compound and excellent stainability (blue fluorescence). It can be apparent from the structure of a basic skeleton of the compound (structure having two benzimidazole rings) that the compound has such effects. Due to the binding affinity for DNA, an X-ray irradiation target atom can be arranged in the close vicinity of DNA. The binding affinity of an iodine atom-containing Hoechst compound for DNA has been confirmed by molecular docking simulation (see, Examples later described). Furthermore, a target cancer cell and the position of its DNA can be visible by stainability. Moreover, Hoechst compounds are highly safe compared to other DNA binding compounds (DNA binding dyes) such as YOYO-1 and Propidium iodide, and thus, can be practically used for treating diseases (administrable to human and non-human animals). Accordingly, the iodine atom-containing Hoechst compound can be used as a DNA targeting substance.

Similarly, a compound (a compound represented by formula (I) or formula (II)), which is modified in structure from a Hoechst compound within the range defined by formula (I) or formula (II), can exhibit a high binding affinity for DNA, excellent stainability (blue fluorescence) and high safety. More specifically, the compound of the present invention binds to DNA to allow an X-ray irradiation target atom to arrange in the close vicinity of DNA; makes the locations of cancer cells and DNA visible due to its stainability; and can be safe even if it is administered to organisms (human and non-human animals). The compound of the present invention can be used by taking the properties such a DNA binding ability into consideration. On the other hand, as described later, an iodine atom, gadolinium atom, gold atom, silver atom and platinum atom are atoms which can easily eject Auger electrons by X-ray irradiation. The present invention is characterized in that high binding affinity of a compound represented by formula (I) or formula (II) for DNA and the Auger effect of an iodine atom and a gadolinium atom are used, and that the Auger therapy can be extremely effectively performed.

Examples of the compounds (analog or derivative) having a structure obtained by modifying the structures of Hoechst compounds will be shown below.

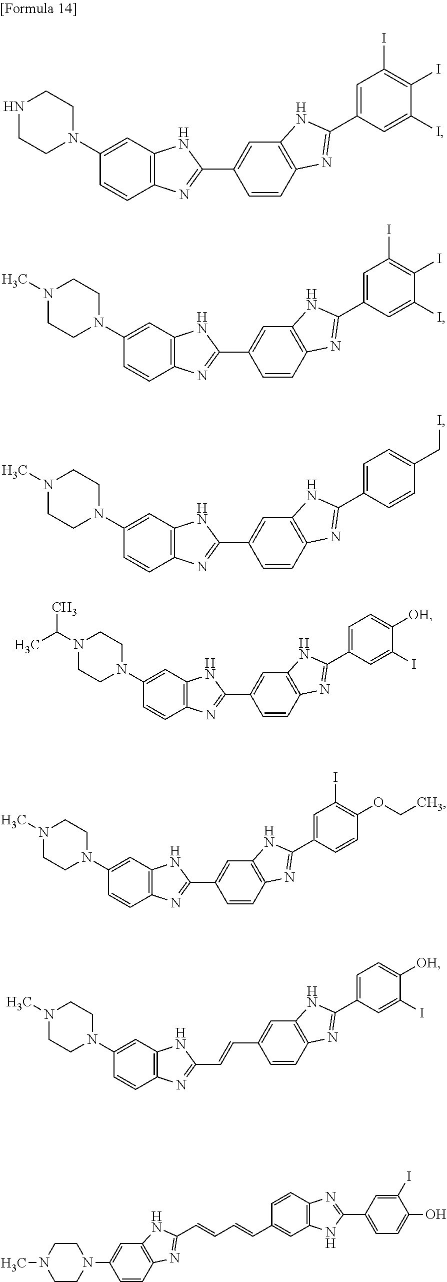

The compound represented by formula (I) or formula (II) except for the commercially available compounds mentioned above can be synthesized from a Hoechst compound as mentioned above and an iodine atom-containing Hoechst compound by, e.g., derivatization. Such a compound can be synthesized by modifying a commonly known synthesis process for a Hoechst compound. As a derivatization method for a Hoechst compound, for example, methods described in the following documents can be appropriately used.
Shrivastava, N., Naim, M. J., Alam, M. J., Nawaz, F., Ahmed, S., & Alam, O. (2017). Benzimidazole Scaffold as Anticancer Agent: Synthetic Approaches and Structure-Activity Relationship. Archiv der Pharmazie, 350(6), 10.1002/ardp.201700040. https://doi.org/10.1002/ardp.201700040
Di Gioia, M. L., Cassano, R., Costanzo, P., Herrera Cano, N., Maiuolo, L., Nardi, M., Nicoletta, F. P., Oliverio, M., & Procopio, A. (2019). Green Synthesis of Privileged Benzimidazole Scaffolds Using Active Deep Eutectic Solvent. Molecules (Basel, Switzerland), 24(16), 2885. https://doi.org/10.3390/molecules24162885
Balagurumoorthy, P., Xu, X., Wang, K., Adelstein, S. J., & Kassis, A. I. (2012). Effect of distance between decaying (125)I and DNA on Auger-electron induced double-strand break yield. International journal of radiation biology, 88(12), 998-1008. https://doi.org/10.3109/09553002.2012.706360

### Nanoparticle

The present invention also relates to a nanoparticle comprising a compound represented by formula (I) or a pharmaceutically acceptable salt thereof and a porous silica carrier. Furthermore, the present invention relates to a nanoparticle containing an iodine atom (IPO). Note that, for IPO, see Non Patent Literature 2.

More specifically, the nanoparticle of the present invention can be a particle constituted of a porous silica carrier loaded with a compound represented by formula (I) or a pharmaceutically acceptable salt thereof. A preferred aspect of a compound represented by formula (I) is as explained above and the whole explanation applies to the compound to be used in the nanoparticle. For example, a compound represented by formula (I) is preferably a compound represented by formula (II) and more preferably an iodine atom-containing Hoechst compound (particularly any one of four compounds).

The porous silica carrier is a carrier formed of porous silica (a substance capable of carrying a compound) and the following porous silica can be used.

Porous silica is a substance having silicon dioxide (silica: SiO₂) as a main component and a number of pores. Porous silica may have a particle form. A main component (most abundant component) constituting a nanoparticle can be constituted of porous silica. Porous silica is characterized by a large specific surface area due to the presence of pores. Porous silica can be a nanoparticle. As used herein, the nanoparticle refers to a nano-size particle. A porous silica nanoparticle may serve as a support and/or a substrate for carrying the compound of the present invention. As used herein, the nano-size usually refers to 10 nm or more and 500 nm or less, and preferably 40 nm or more and 400 nm or less.

In the nanoparticle of the present invention, the compound of the present invention can be present on the surface of porous silica (the outer surface of a particle and inner surface in a pore). As an aspect of porous silica carrying the compound of the present invention, the compound may bind to porous silica. The binding of the compound to porous silica may be a chemical binding or an electrical binding. Silica may have a silanol group (Si—OH) or a group derived from a silanol group on the surface thereof. The compound may be bound to silica via the silanol group. On the other hand, the compound of the present invention has a nitrogen atom and an unshared electron pair may be present in the nitrogen atom. Silica may be bound to silica via the unshared electron pair. Alternatively, silica and the compound of the present invention may be mutually electrically attracted to bind (attach) via electrostatic force. The aspect of porous silica carrying the compound of the present invention is not particularly limited as long as it satisfies the object and purpose of the present invention.

In a preferred embodiment, porous silica is mesoporous silica. The mesoporous silica usually has a number of pores having a pore size (pore diameter) of 2 to 50 nm. Since the mesoporous silica has a larger specific surface area, a compound as mentioned above can be further efficiently loaded. As described later, mesoporous silica has an advantage in that it is easily taken up by a cell. The porous silica described herein can be all replaced with mesoporous silica unless otherwise specified.

In a preferred specific embodiment, a nanoparticle (silica carrier) may be a biodegradable mesoporous silica nanoparticle. A method for synthesizing biodegradable mesoporous silica is later described, for example, in [Examples], Production Example 1 (paragraph [0091]). The biodegradable mesoporous silica can be decomposed in vivo with the passage of time. Examples of the mechanism of decomposition include an enzymatic reaction. In the case of biodegradable mesoporous silica, a nanoparticle can be decomposed in vivo and components thereof can be excreted. For this reason, treatments and the like can be more safely performed. The biodegradable mesoporous silica can be obtained from a silane compound as a raw material having a biodegradable structure. Examples of the biodegradable structure include bonds represented by S—S and/or S—S—S—S. For example, bis[3-(teriethoxysilyl)propyl]tetrasulfide is a silane compound having an S—S—S—S bond between two Si atoms. When this compound is incorporated into a mesoporous silica structure, a structure having an S—S—S—S bond between two Si atoms may be formed in mesoporous silica. Since S—S and S—S—S—S have relatively weak binding force, they are easily biodegraded.

In a nanoparticle, the ratio of the compound of the present invention to porous silica (the compound of the present invention/porous silica) may vary depending on the type of X-ray irradiation target atom. Although, not particularly limited, the ratio by weight falls, for example, within the range from 0.0001 to 1, preferably, 0.001 to 0.5, and more preferably 0.001 to 0.1. The ratio of the compound of the present invention to porous silica may vary depending on the ratio of an X-ray irradiation target atom such as an iodine atom to porous silica. Although is not particularly limited, the ratio of the X-ray irradiation target atom to porous silica (X-ray irradiation target atom/porous silica) falls, for example, within the range of 0.00001 to 1, preferably 0.0001 to 0.5 and more preferably 0.0001 to 0.1. The ratio (X-ray irradiation target atom/porous silica) is particularly preferably 0.0005 or more. The weight of X-ray irradiation target atom in a nanoparticle can be obtained by analyzing the X-ray irradiation target atom in the nanoparticle by an inductively coupled plasma optical emission spectroscopy (ICP-AES).

As the porous silica, a synthesized porous silica can be used. A synthesis method for porous silica will be described later, for example, in [Examples], Production Example 1 (paragraph [0091]). The synthesis method for porous silica is not particularly limited and a commonly known method can be used. For example, a precursor substance (for example, an organosilane compound, more specifically an alkylalkoxysilane) for forming porous silica is condensed in the presence of a template compound for forming pores (for example, tetraalkoxysilane, more specifically tetraethoxysilane), and the template compound is removed from the silica particles formed by the condensation to obtain a porous silica particle (see, for example, International Publication No. WO 2021/060498). Examples of the precursor substance of porous silica include 1,2-bis(triethoxysilyl)ethane. Examples of the template compound include cetyltrimethylammonium bromide (CTAB).

Loading of the present invention compound into porous silica can be performed, for example, by mixing the compound of the present invention and porous silica in an appropriate solvent (for example, adding porous silica particles in a solution having a compound dissolved therein and suspending them) but not limited to this. When the compound comes in contact with porous silica, the compound is loaded into the porous silica by binding as mentioned above (chemical or electrical binding). In the mixing operation, a rotary mixer can be used. After the mixing operation, the mixture is, for example, centrifuged. After the supernatant is removed, washing is performed several times with a washing solution (for example, water) to remove an excessive compound. In this manner, the nanoparticle of the present invention having porous silica carrying the compound of the present invention can be obtained. An iodine-containing compound (for example, Propidium Iodide) other than IH can be loaded by the above method and used for the Auger therapy.

The nanoparticle of the present invention has a feature in that it can be easily taken up by a cell, particularly, a cancer cell. It has been confirmed that nanoparticles enter the cells when brought into contact with cells. According to a hypothesis, it is presumed that a cell uses endocytic machinery involving endosomal vesicles to take up a nanoparticle. The endosomal vesicles may deliver the nanoparticle to lysosomes localized adjacent to the nucleus of a cell. Needless to say, the present invention is not limited by the hypothesis. Accordingly, the nanoparticle of the present invention can deliver the compound of the present invention to a site near the nucleus of a cell.

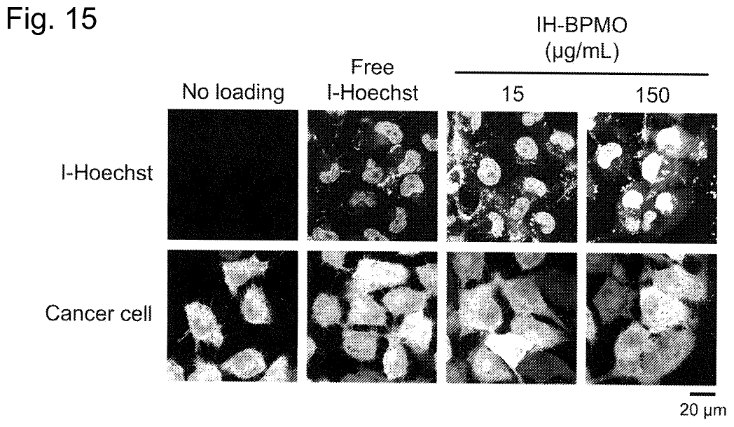

The nanoparticle of the present invention is useful for targeting to solid cancer such as a tumor. When a nanoparticle is administered to a human or an animal, there is a possibility that the nanoparticle accumulates in solid cancer. Because of this, the nanoparticle has at least two advantages, i.e., reaching solid cancer and taking up by a cancer cell. Furthermore, after the nanoparticle of the present invention is taken up by a cancer cell, the compound of the present invention (a compound represented by formula (I), more specifically, for example, iodine atom-containing Hoechst compound) can be released from the nanoparticle in the conditions such as low pH of intracellular endosomes, and migrate into the nucleus of the cell to bind to DNA. More specifically, the nanoparticle of the present invention is capable of accumulating in a tumor and delivering an X-ray irradiation target atom such as an iodine atom into DNA in a cancer cell. Since targeting can be made as mentioned above, it is possible to prevent side effects by systemic administration (see, FIG. 15).

As mentioned above, the compound of the present invention has an analogous chemical structure to a dye for Hoechst stain and excellent binding ability to the nucleus of a cell, and simultaneously has an atom ejecting Auger electrons such as an iodine atom (X-ray irradiation target atom) upon X-ray irradiation. Because of this, owing to the nanoparticle, the compound of the present invention can be arranged in the vicinity of a cancer cell and efficiently taken up by the nucleus of a cell, with the result that the Auger therapy can be extremely effectively performed.

### Pharmaceutical Composition

According to an aspect of the present invention, the present invention relates to a pharmaceutical composition containing the compound of the present invention and/or a nanoparticle of the present invention, for use in radiotherapy.

The pharmaceutical composition contains the compound of the present invention explained above and a pharmaceutically acceptable carrier. The pharmaceutically acceptable carrier may be in the form of a liquid or a solid. The carrier may be, e.g., an excipient, a diluent or an adjuvant. Examples of the liquid carrier include water and an organic solvent. Examples of the organic solvent include, but are not limited to, alcohol solvents such as methanol and ethanol; ketone solvents such as acetone; ether solvents such as diethyl ether and ester solvents such as ethyl acetate. Examples of the solid carrier include lactose, crystalline cellulose and starch. Note that, the carriers mentioned herein are just examples. A carrier commonly known in the technical field can be appropriately used in the pharmaceutical composition.

In a specific embodiment, the pharmaceutical composition contains the nanoparticle of the present invention and a pharmaceutically acceptable carrier. The nanoparticle carries the compound of the present invention and the pharmaceutical composition contains the compound of the present invention loaded in a nanoparticle.

A pharmaceutical composition can be irradiated with X-rays as radiation. As mentioned above, the X-ray irradiation target atom (iodine atom) in the compound of the present invention can eject electrons due to the Auger effect upon irradiation of X-rays. Owing to the pharmaceutical composition of the present invention, the compound can easily arrive at a target site.

The pharmaceutical composition can be used for treating solid cancer or suppressing the enlargement or growth of solid cancer. As described above, Auger electrons ejected from an X-ray irradiation target atom (e.g., iodine atom) upon irradiation of X-rays can destroy cancer cells. Accordingly, the pharmaceutical composition is useful for treating solid cancer and suppressing the enlargement or growth of solid cancer.

Examples of the solid cancer include, but are not limited to, a brain tumor, lung cancer, ovarian cancer, digestive system cancer, osteosarcoma and head and neck cancer.

The solid cancer may be in a hypoxic condition. In the present invention, solid cancer in a hypoxic condition can be efficiently treated. The existing X-ray irradiation mostly use reactive oxygen. However, in a hypoxic condition, reactive oxygen is not sufficiently generated, so that a therapeutic effect cannot be always sufficiently obtained. Whereas, the radiotherapy according to the present invention depends on not reactive oxygen but the Auger effect. Because of this, cancer can be effectively treated even in a hypoxic condition as already confirmed (see, Examples later described). Accordingly, the present invention makes it possible to effectively treat cancer even in hypoxic conditions under which existing radiotherapies rarely produce effects.

The pharmaceutical composition can be administered by an appropriate administration method. The administration method may be an oral administration or a parenteral administration. Examples of the parenteral administration include an injection (e.g., intravenous injection, subcutaneous injection, intramuscular injection), a suppository administration and an external application (skin application, mucosa application). The dosage of the pharmaceutical composition for radiotherapy is not particularly limited but it is preferably determined by the amount of the composition that sufficiently produces the Auger effect when the compound of the present invention is irradiated with X-rays.

### Radiotherapy (X-Ray Irradiation) and Cancer Cell Destruction

Now, the radiotherapy (X-ray irradiation) according to the present invention will be explained. The following explanation can apply to the compound, nanoparticle and pharmaceutical composition of the present invention.

According to an aspect of the present invention, the present invention relates to irradiation of the compound of the present invention (including the case where the compound is loaded in nanoparticle) with an X-ray, and cancer cell destruction caused by the X-ray irradiation. More specifically, an X-ray irradiation target atom (more specifically, an iodine atom, a gadolinium atom, a gold atom, a silver atom or a platinum atom) can be irradiated with X-rays. Auger electrons can be ejected from the X-ray irradiation target atom (e.g., iodine atom) by irradiation with an X-ray that can excite the K shell electrons of the X-ray irradiation target atom.

Generally, Auger electrons have a possibility of causing damage to DNA and other cell components. The X-ray irradiation target atom is suitable for ejection of Auger electrons. However, Auger electrons reach in a limited range. Because of this, a cell destructive effect by Auger electrons has not been sufficiently demonstrated in previous research. The present invention has an advantage in that the X-ray irradiation target atom is arranged in the vicinity of the nucleus (particularly DNA) of a cancer cell by use of a compound having a predetermined atom (X-ray irradiation target atom) and a Hoechst-like structure in combination, and the cancer cell is efficiently destroyed by Auger electrons.

When the X-ray irradiation target atom arranged in the vicinity of a nucleus of a cell is irradiated with an X-ray that can excite the K shell electrons of the X-ray irradiation target atom, Auger electrons are ejected from the X-ray irradiation target atom and cause damage to the cell. Since cellular machinery including cell organelles are present in and around the cell nucleus, Auger electrons can damage the machinery, resulting in damaging cells efficiently and effectively. Subsequently, the cell damage leads to the destruction or death of the cancer cell.

The X-ray that can excite the K shell electrons of an X-ray irradiation target atom varies depending on the X-ray irradiation target atom, and the energy level and/or wavelength intrinsic to the X-ray irradiation target atom are present. The X-ray can be an X-ray having an energy level that can excite the K shell electrons. Furthermore, the X-ray can be an X-ray having a wavelength that can excite the K shell electrons. Based on the “International tables for crystallography C, Table 4.2.2.4 theoretical calculations” disclosed by the International Union of Crystallography (IUCr), K shell electron excitation wavelengths (corresponding X ray wavelengths) and the energy for exciting the K shell electron of I, Gd, Au, Ag and Pt are as listed in Table 1.

TABLE 1

| | λ (Å) | λ (nm) | E (keV) |
|---|---|---|---|
| I | 0.3736775 | 0.03737 | 33.17 |
| Gd | 0.2467265 | 0.02467 | 50.25 |
| Au | 0.1535699 | 0.01536 | 80.73 |
| Ag | 0.4857609 | 0.04858 | 25.52 |
| Pt | 0.1581346 | 0.01581 | 78.40 |

In the case of an iodine atom, an X-ray having an energy of 33.2 keV is suitable for exciting the K shell electron. This is because the energy for exciting the K shell electrons of an iodine atom is 33.2 keV (more precisely, 33.17 keV). However, even if the energy level is not optimum, K shell electrons may be excited. It has been confirmed that an X-ray of 33.4 keV in energy is effective in iodine atoms. Accordingly, when an iodine atom is irradiated with an X-ray of 33.2 keV or 33.4 keV in energy, Auger electrons can be ejected from the iodine atom. Note that, the wavelength of the X-ray having an energy of 33.17 keV that can excite K shell electrons of an iodine atom is 0.03737 nm.

Similarly, the energy levels (or wavelength) of X-ray suitable for exciting the K shell electrons of a gadolinium atom, a gold atom, a silver atom and a platinum atom, are 50.25 keV, 80.73 keV, 25.52 keV and 78.4 keV, respectively. Accordingly, when these X-ray irradiation target atoms are irradiated with X-rays having different energy levels (or wavelengths), Auger electrons can be ejected.

Note that, the K shell electron excitation energy is also called as the K shell absorption-edge energy, and the K shell electron excitation wavelength is also called as the K shell absorption-edge wavelength.

The X-ray may be an X-ray having a spectral peak preferably at E−0.5 keV or more, more preferably E−0.3 keV or more, and further preferably E−0.1 keV or more to the K shell electron excitation energy E of an X-ray irradiation target atom. The X-ray may be an X-ray having a spectral peak preferably at E+0.8 keV or less, more preferably E+0.7 keV or less, further preferably E+0.6 keV or less and further more preferably E+0.5 keV or less to the K shell electron excitation energy E of an X-ray irradiation target atom. More specifically, the X-ray may be an X-ray having a spectral peak preferably at any of E−0.5 keV or more, E−0.3 keV or more, E−0.1 keV or more and E−0.05 keV or more, and a spectral peak at any of E+0.8 keV or less, E+0.7 keV or less, E+0.6 keV or less and E+0.5 keV or less. Particularly, the X-ray is preferably an X-ray having a peak in the range of E−0.1 keV or more and E+0.5 keV or less to the K shell electron excitation energy E of an X-ray irradiation target atom. As mentioned above, the value of E varies depending on each of the X-ray irradiation target atoms. Auger electrons can be efficiently ejected by irradiation with an X-ray having the energy corresponding to the X-ray irradiation target atom. Even irradiation with an X-ray having an energy level in the vicinity of the K shell electron excitation energy E of an X-ray irradiation target atom may have the Auger effect. In particular, even irradiation with an X-ray having slightly higher energy than K shell electron excitation energy E can have the Auger effect. Because of this, an energy of E+0.5 keV or less is preferable. In contrast, it is difficult to obtain the Auger effect at an energy lower than K shell electron excitation energy E. Because of this, an energy of E−0.1 keV or more is preferable. From the above description, it can be understood that an energy of 33.1 to 33.7 keV is preferable in the case of an iodine atom (E=33.2 keV).

The X-ray is preferably a monochromatic X-ray or a characteristic X-ray. Furthermore, the X-ray is more preferably a monochromatic X-ray. The monochromatic X-ray refers to an X-ray having a very narrow energy range. As used herein, a monochromatic X-ray of energy E; is defined that the spectral peak of monochromatic X-ray is present at a position of energy E₁ and does not include an X-ray having an energy of e.g., E₁−ΔE keV or less and E₁+ΔE keV or more. Herein, ΔE satisfies ΔE≤E×10⁻³. The characteristic X-ray is defined as excessive energy generated when electrons are transferred from the outer shell into holes, which are produced by excitation of the inner shell, and emitted as the X-ray. The energy of the characteristic X-ray is determined by the difference in energy between the inner shell and the outer shell, that is, a value intrinsic to a material. Accordingly, it is difficult to take out a monochromatic X-ray having arbitral energy. Since the monochromatic X-ray and characteristic X-ray have narrow energy range, unlike continuous X-ray (or white X-ray), it is possible to effectively reduce the degree of damage given to normal cells and tissues caused by X-ray irradiation. In particular, a monochromatic X-ray is excellent in such an effect. A monochromatic X-ray can be taken out by monochromatizing white X-ray generated by a synchrotron radiation facility or an X-ray generator, by a spectrometer. Whereas the characteristic X-ray can be taken out by monochromatizing only characteristic X-ray contained in white X-ray generated by an X-ray generator, by a spectrometer. However, methods for generating a monochromatic X-ray and a characteristic X-ray are not limited to these.

As shown in Examples later described, when an iodine atom was used as the X-ray irradiation target atom, cancer cells were successfully destroyed by irradiation with an X-ray having an energy of 33.2 keV. Furthermore, irradiation with an X-ray having an energy of 33.4 keV successfully destroyed cancer cells even though effectiveness is inferior to the X-ray of 33.2 keV. In contrast, irradiation with X-ray of 33.0 keV did not virtually destroy cancer cells. Dramatic difference in effect between the X-ray of 33.0 KeV and the X-ray of 33.2 keV supports the view that Auger electrons effectively destroy cells. When the X-ray obtained by a commercially available X-ray irradiation device for experiments and researches is used, radio (X-ray) sensitization effect is observed.

According to an aspect of the present invention, the present invention relates to a method for treating solid cancer or suppressing enlargement or growth of solid cancer. The method includes irradiating with an X-ray the compound, nanoparticle or pharmaceutical composition taken in a body of a subject, to destroy a cancer cell. As the X-ray, an X-ray that can excite the K shell electrons of an X-ray irradiation target, can be used. The mechanism of how to destroy a cancer cell is as explained above. Examples of the subject include patients. Note that, the method can be applied, needless to say, to humans as well as non-human animals.

The irradiation time of an X-ray may vary depending on the severity of the disease to be treated or the tolerance of the patient to the X-ray irradiation but is not particularly limited. The irradiation time can be set at, for example, 1 minute or more, 2 minutes or more, 3 minutes or more or 5 minutes or more. Furthermore, the irradiation time of an X-ray is not particularly limited but can be set at, for example, 240 minutes or less, 180 minutes or less, 150 minutes or less or 120 minutes or less. For example, the irradiation time of an X-ray may be 10 minutes, 30 minutes, 60 minutes or 90 minutes.

As shown in Examples later described, the compound of the present invention is excellent in targeting solid cancer and can easily permeate into the nucleus of a cancer cell. When the compound of the present invention irradiated with an X-ray, the cancer cell can be destroyed by Auger electrons. Accordingly, solid cancer is effectively treated or enlargement or growth of solid cancer can be effectively suppressed by this method.

### EXAMPLES

Now, the present invention will be more specifically described by way of Examples but, needless to say, the present invention is not limited to Examples.

### Materials (Compounds)

As the iodine atom-containing Hoechst compounds, the following commercially available iodine atom-containing Hoechst compounds were obtained.
Hoechst 33342 analog 2 trihydrochloride (manufactured by MedChemExpress)
meta-iodoHoechst 33258 (manufactured by MedChemExpress)
para-iodoHoechst 33258 (manufactured by MedChemExpress)
ortho-iodoHoechst 33258 (manufactured by MedChemExpress)

### Test Example 1

### Docking Simulation Between Iodine Atom-Containing Hoechst Compound and DNA

Docking simulation between an iodine atom-containing Hoechst compound and DNA was performed by use of a molecular docking method.

As the iodine atom-containing Hoechst compound, four compounds: “Hoechst 33342 analog 2”, “meta-iodoHoechst 33258”, “para-iodoHoechst 33258” and “ortho-iodoHoechst 33258”, were used.

To obtain DNA for use in docking, a typical B-DNA was constructed using software “3DNA” (see, Lu X J, Olson W K. 3DNA: a software package for the analysis, rebuilding and visualization of three-dimensional nucleic acid structures. Nucleic Acids Res. 2003 Sep. 1; 31 (17): 5108-21. doi: 10.1093/nar/gkg680).

As the software for docking simulation, AutoDock4 was used (see, Morris G M, Huey R, Lindstrom W, Sanner M F, Belew R K, Goodsell D S, Olson A J. AutoDock4 and AutoDockTools4: Automated docking with selective receptor flexibility. J Comput Chem. 2009 December; 30 (16): 2785-91. doi: 10.1002/jcc.21256).

An iodine atom-containing Hoechst compound serving as a ligand was completely flexibly docked with DNA.

Of the 20 states of binding predicted, the state of binding to a target binding site and having a low estimated free energy of binding (ΔG) was selected.

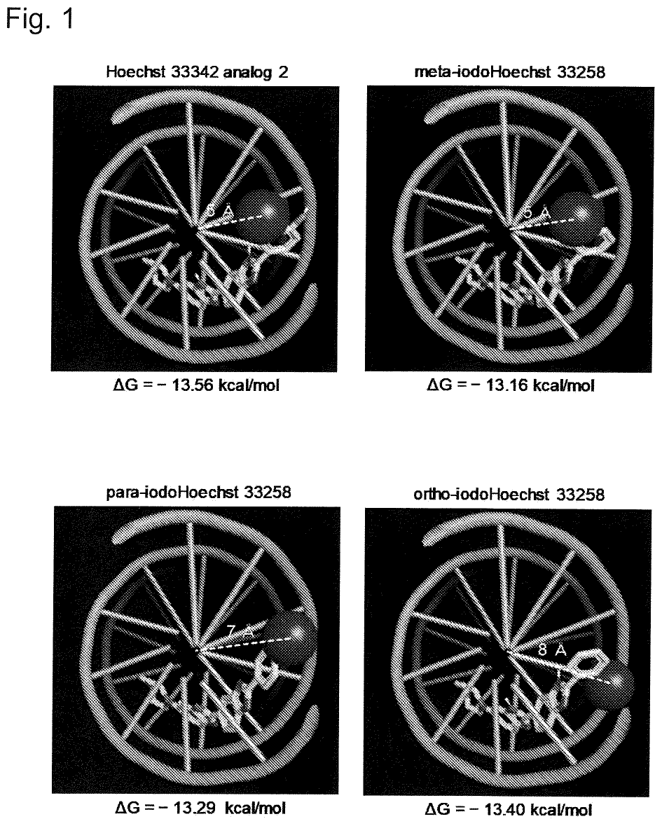

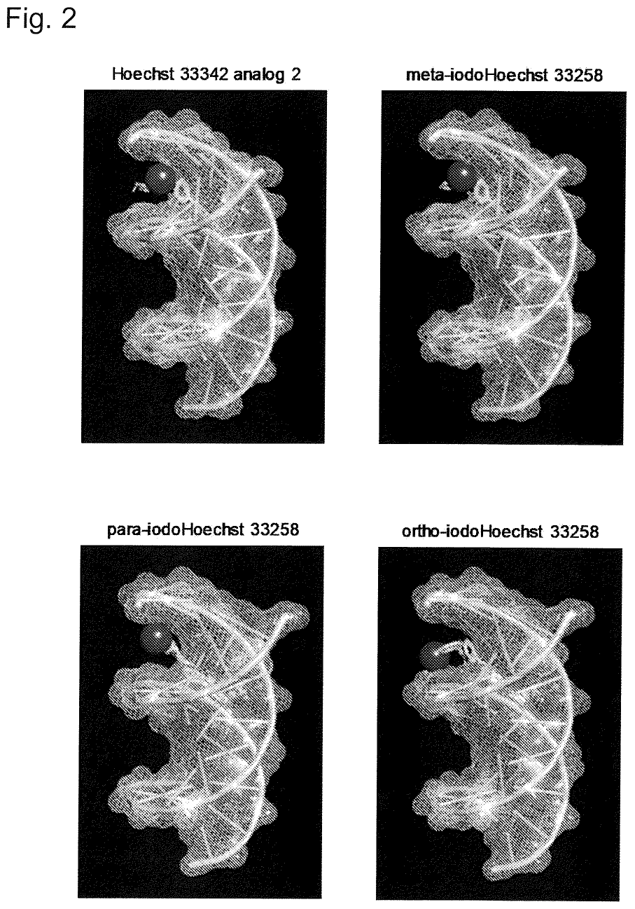

The results of the docking simulation (images and calculation values) are shown in FIG. 1, FIG. 2 and Table 2.

TABLE 2

| | Estimated free | Distance between |
|---|---|---|
| | energy of | iodine atom and |
| | binding (ΔG) | center of DNA |
| Hoechst 33342 analog 2 | −13.56 kcal/mol | 5 Å |
| meta-iodoHoechst 33258 | −13.16 kcal/mol | 5 Å |
| para-iodoHoechst 33258 | −13.29 kcal/mol | 7 Å |
| ortho-iodoHoechst 33258 | −13.40 kcal/mol | 8 Å |

FIG. 1 shows a view along the axis of a helix (top view) and FIG. 2 is a view perpendicular to the axis of the helix viewed along the axis of the helix (side view).

As shown in these figures and the Table, it was confirmed by molecular docking simulation that the four iodine atom-containing Hoechst compounds all have high binding affinity for DNA. Accordingly, it was suggested that an X-ray irradiation target atom (iodine atom) to be in the close vicinity of DNA can be arranged by iodine atom-containing Hoechst compounds.

Herein, it was shown that “Hoechst 33342 analog 2” of the four iodine atom-containing Hoechst compounds, has the smallest estimated free energy of binding (ΔG) (−13.56 kcal/mol); high binding affinity for DNA; and iodine is close to the center of DNA (5 Å). Thus, it is presumed that “Hoechst 33342 analog 2” is the most favorable of the four compounds. Then, in the following experiments, “Hoechst 33342 analog 2” (hereinafter also referred to as “I-Hoechst”) was mainly investigated as the iodine atom-containing Hoechst compound. However, in consideration of availability, a trihydrochloride of “Hoechst 33342 analog 2”, that is, “Hoechst 33342 analog 2 trihydrochloride” (hereinafter also referred to as “I-Hoechst-3HCl”) was used.

### Test Example 2

### Uptake of Iodine Atom-Containing Hoechst Compound by Cancer Cell

Human ovarian cancer cell OVCAR8 expressing green fluorescent protein (GFP) was used as a cancer cell. Cancer cell OVCAR8 was cultured on RPMI1640 medium containing 10% inactivated FBS and 1% penicillin/streptomycin in a culture dish having a diameter of 100 mm. Furthermore, the iodine atom-containing Hoechst compound (I-Hoechst-3HCl) (0.1 mg, 151.57 nmol) was dissolved in water (0.15 mL) to prepare a solution (the amount of Hoechst compound: 1 μmol/mL) containing the iodine atom-containing Hoechst compound. The solution was added to the culture solution such that the amount of the Hoechst compound in the culture solution became 50 nmol/mL and incubated in a CO₂ incubator at 37° C. for 24 hours. Thereafter, the culture medium was removed and the cells were washed. The cells were observed by a confocal microscope. Cell nuclei can be detected based on staining with a Hoechst compound (blue color emission) and expression of GFP (green fluorescence).

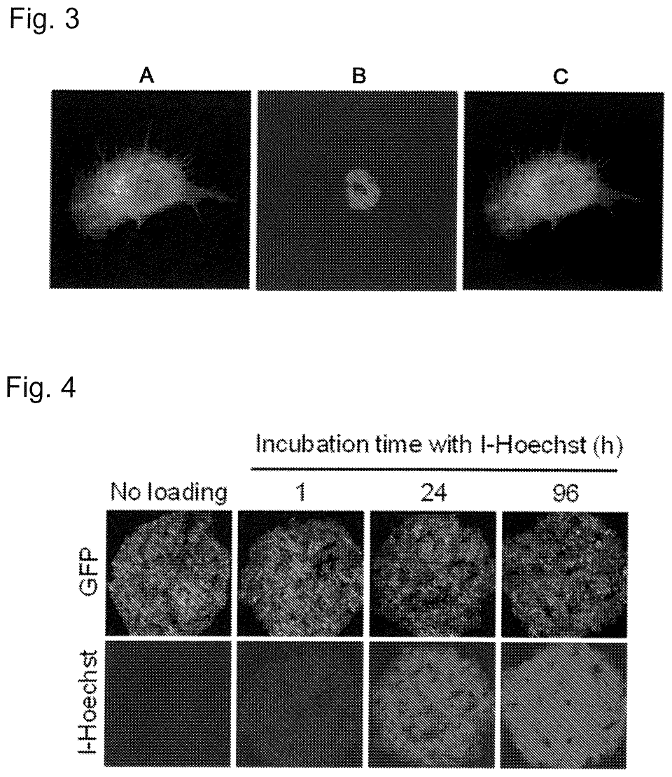

FIG. 3 shows images observed by a microscope. The figure shows a typical cell. FIG. 3A shows cancer cell OVCAR8 emitting fluorescence (green). FIG. 3B shows a region (blue) stained with the Hoechst compound serving as a dye; and Figure C shows a processed image obtained by superimposing FIG. 3A and FIG. 3B.

From FIGS. 3A to C, it is confirmed that the region stained with the Hoechst compound is present in the interior of the cancer cell, and found that the Hoechst compound is taken up within the cell, transported to the nucleus of the cell and localized in DNA.

### Test Example 3

Uptake of Hoechst Compound into Tumor Spheroid

Human ovarian cancer cell OVCAR8 expressing green fluorescent protein (GFP) was used as a cancer cell. A tumor spheroid (hereinafter also referred to as “spheroid”) was formed from the cancer cell. Cancer cell OVCAR8 was cultured on RPMI1640 medium containing 10% inactivated FBS and 1% penicillin/streptomycin in a culture dish of 100 mm in diameter. For forming spheroids, 5.0×10 of OVCAR8 cells were seeded in PrimeSurface96U culture plate (MS-9096U, Sumitomo Bakelite Co., Ltd). The OVCAR8 cells were cultured in a CO₂ incubator at 37° C. for 7 days. Since the cells were not able to attach to a hydrophilic plate surface, the cells came together at the bottom of a well and formed three-dimensional spheroids at the bottom. In this manner, spheroids (estimated number of cells: approximately 100,000) of 500 μm in diameter were obtained.

Subsequently, an iodine atom-containing Hoechst compound (I-Hoechst-3HCl) (0.1 mg, 151.57 nmol) was dissolved in water (1.5 mL) to prepare a solution containing the iodine atom-containing Hoechst compound (amount of the Hoechst compound: 100 nmol/mL). The solution was added to the spheroids such that the amount of the Hoechst compound became 5 nmol/mL. The mixture was incubated in a CO₂ incubator at 37° C. for a predetermined time.

After incubation, spheroids were collected in an Eppendorf tube, washed with ice-cooled PBS and fixed with 4% paraformaldehyde at 4° C. overnight. The spheroids were washed with ice-cooled PBS and treated with 99.8% methanol at −80° C. for 30 minutes. The spheroid sample thus obtained was observed by a confocal microscope.

FIG. 4 shows results (images) of the microscopic observation on spheroids. For the horizontal row, images in the column indicated by “GFP” are the observation results based on a fluorescence (green), whereas images in the column indicated by “I-Hoechst” are observation results based on staining (blue) with the Hoechst compound. Furthermore, for the vertical row, “No Loading” indicates a sample not treated with the Hoechst compound solution (i.e., control), numerals 1, 24 and 96 indicate the incubation time with the Hoechst compound (1 hour, 24 hours and 96 hours).

As shown in the figure, it was found that the Hoechst compound is efficiently taken up by spheroids and uniformly distributed over the entire spheroids 24 hours later. Interestingly, it was confirmed that the Hoechst compound reaches the interior of a spheroid, by operating a confocal microscope. More specifically, it was confirmed that the Hoechst compound reaches not only cells close to the surface of a spheroid but also cells located at the center of the spheroid. This is different from the initial expectation. Such a high permeability is considered as an advantageous feature of the Hoechst compound.

### Test Example 4

### Monochromatic X-Ray Irradiation to Iodine Atom-Containing Hoechst Compound

### Set Up of Monochromatic X-Ray Radiation Irradiation Device

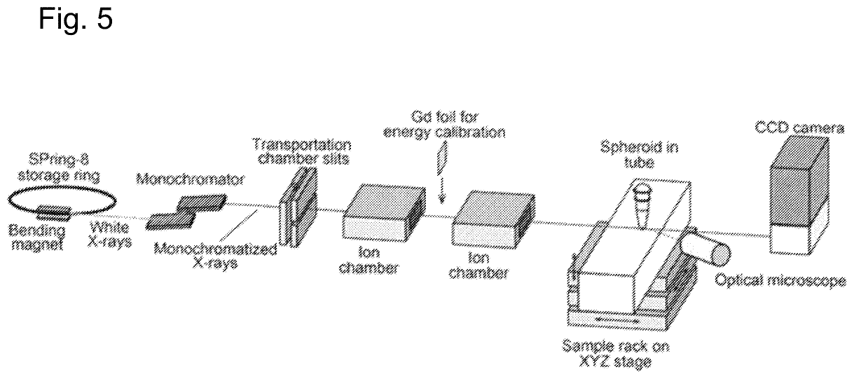

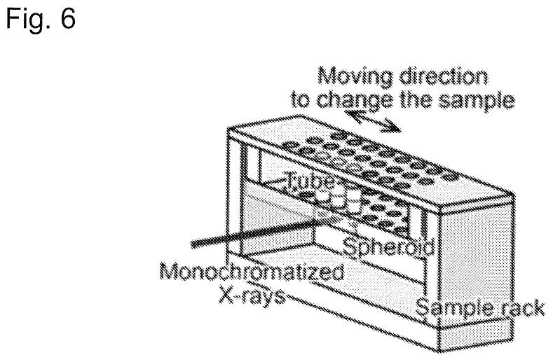

Irradiation of monochromatic X-rays was performed in beamline BL14B1 of large-scale synchrotron radiation facility SPring-8 located in Sayo-cho, Sayo-gun, Hyogo Prefecture, Japan. FIG. 5 schematically shows the setup of the irradiation device. First, white X-ray generated from the bending magnet of SPring-8 was guided to an exit-position fixed two-crystal monochromator having silicon 311 crystal to generate a monoenergetic X-ray beam (monochromatic X-ray). SPring-8 storage ring was operated in the top-up mode under which fluctuation of X-ray intensity over time can be ignored due to a constant storage current of 100 mA to perform an irradiation experiment. The shape of the X-ray beam was controlled by using horizontal and vertical transport channel (TC) slits. The size of the X-ray beam at the position of a sample was 0.7 mm in height×1.4 mm in width. This is large enough to cover a spheroid having a size of 0.5 mm×0.5 mm. During experiments, the intensity of the X-ray beam was monitored in two ion chambers arranged on the optical axis. The X-ray transmitted was monitored by a CCD camera to control the position of the sample. FIG. 6 schematically shows a rack of spheroid samples. Spheroids are placed at the bottom of tubes put in a sample rack.

### Test Example 5

### Destruction of Spheroid by Monochromatic X-Ray Irradiation

The iodine atom-containing Hoechst compound (I-Hoechst) and spheroids were incubated (24 hours) in the same manner as in Test Example 3 to obtain spheroids having the Hoechst compound taken up. The spheroids were put in tubes and the tubes were plated at the sample rack of the X-ray irradiation device. Since the sample rack is designed such that samples can be moved on the XYZ stage, the experimenter can perform X-ray irradiation by moving samples on the optical axis without entering the experimental hutch. After completion of X-ray irradiation to a sample, the sample rack is designed to automatically move such that the irradiation position faces the next sample. Thus, a series of X-ray irradiation can be automatically performed (see, FIG. 6). Before irradiation, the position of a sample was confirmed by an optical microscope and a laser, and monitored by a CCD camera during X-ray irradiation. Note that, since the energy of X-ray is extremely high, observation of spheroids and an absorption control of a tube by the CCD camera was not made. A refractive X-ray image of a tube was obtained to monitor of a sample position. The photon flux at a position of a sample was calculated as 3.9×10⁸ (photons/second) by use of the SPECTRA code.

The spheroids prepared as mentioned above were irradiated with a monochromatic X-ray (energy: 33.0 key, 33.2 keV or 33.4 keV) for 30 minutes. After X-ray radiation, spheroids were incubated in a CO₂ incubator at 37° C. for 3 days and thereafter, observed by a confocal microscope (visible light and fluorescent light). For comparison, an iodine-free Hoechst compound (Hoechst 33258, Dojindo (manufactured by Dojindo Laboratories)) was subjected to the same treatment (incubated with spheroids, irradiated with monochromatic X-rays followed by incubation).

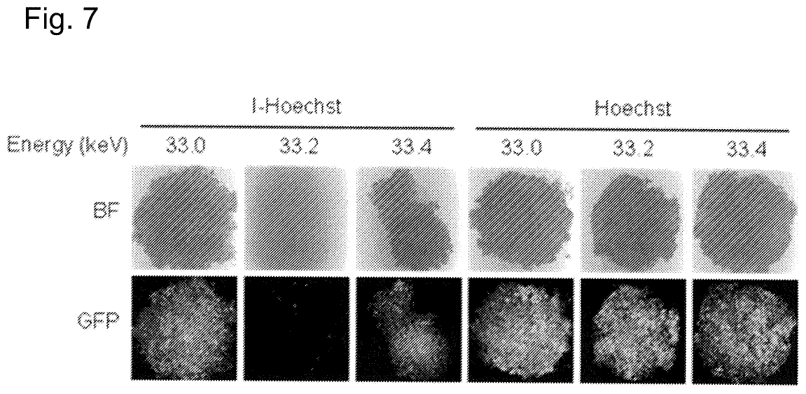

FIG. 7 shows the results (images) of microscopic observation on spheroids after irradiation with monochromatic X-rays followed by incubation. For the horizontal row, images in the column indicated by “BF” are the results based on the bright field and images in the column indicated “GFP” are the observation results of a fluorescence (green). The vertical row shows the energy values of the monochromatic X-rays. As shown in the figure, when spheroids having an iodine atom-containing Hoechst compound taken up were irradiated with a monochromatic X-ray having an energy of 33.2 keV, the presence of spheroids were not observed either in the bright field (BF) or GFP. Accordingly, it was demonstrated that spheroids having the iodine atom-containing Hoechst compound taken up and irradiated with the monochromatic X-ray having an energy of 33.2 keV for 30 minutes are destroyed into pieces. Furthermore, it was found that the number of spheroids decreased by irradiation of the monochromatic X-ray of 33.4 keV and spheroids were destroyed even though the degree of destruction was lower than the 33.2-keV monochromatic X-ray. In contrast, in the case of irradiation with the monochromatic X-ray of 33.0 keV, the number of spheroids did not decrease, and thus, spheroids were not destroyed. Note that, when the spheroids having an iodine-free Hoechst compound taken up therein were irradiated with any of monochromatic X-rays different in energy, spheroids were not destroyed. The energy values mentioned above correspond to the K shell electron excitation energy values of an iodine atom. It was suggested that cells were destroyed by the Auger effect.

For investigating the mechanism of how to destroy spheroids, spheroids were irradiated with a monochromatic X-ray for 30 minutes. Immediately after irradiation and before incubation, spheroids were subjected to γH2AX assay to confirm the state of a DNA double strand. In the γH2AX assay, if a double strand is cleaved, red fluorescence is emitted. The γH2AX assay was performed by use of a commercially available kit in accordance with the instructions attached thereto.

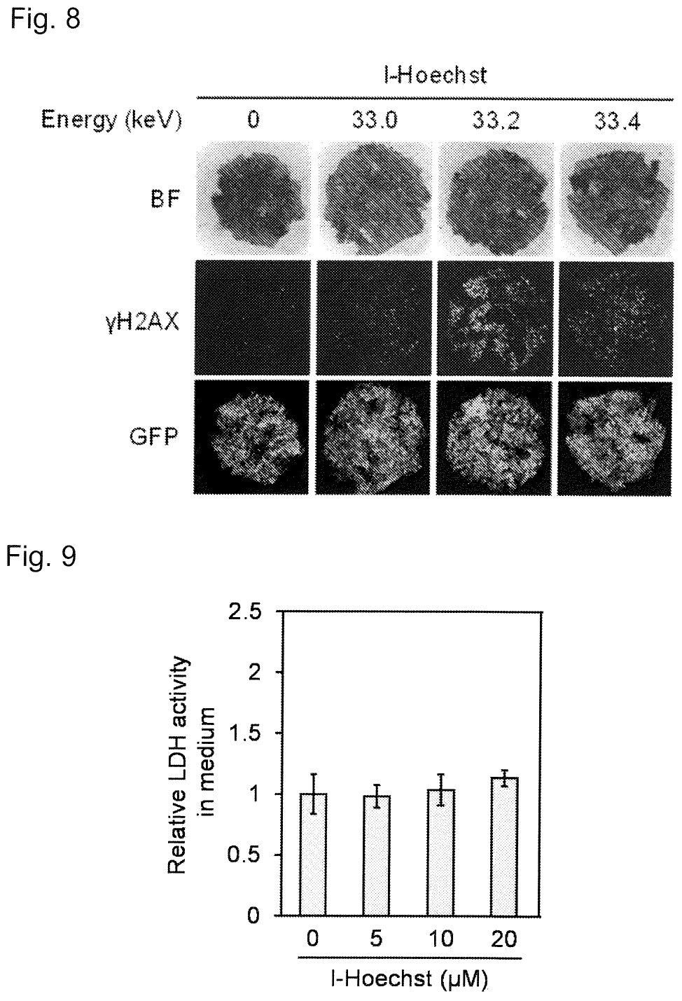

FIG. 8 shows the results (images) of microscopic observation on spheroids after irradiation with monochromatic X-rays (immediately after the irradiation). For the horizontal row, images in the column indicated by “BF” are the results based on the bright field; images in the column indicated by “γH2AX” are the results based on a fluorescence (red); and images in the column indicated by “GFP” are the results based on a fluorescence (green). Furthermore, the vertical row shows the energy values of the monochromatic X-rays (note that, “0” means not irradiated). As shown in the figure, when spheroids having iodine atom-containing Hoechst compound taken up were irradiated with 33.2-keV monochromatic X-ray, red fluorescence was observed. This meant that a DNA double strand was broken (note that, since the time point is before incubation, green fluorescence (GFP) derived from cancer cells is observed). Also in the case of irradiation with the 33.4-keV monochromatic X-ray, it was found that a DNA double strand was broken although the degree of double strand break is lower than the 33.2-keV monochromatic X-ray. In contrast, in the case of irradiation with the 33.0-keV monochromatic X-ray and the case of no irradiation with a monochromatic X-ray, no DNA double strands were broken. These results demonstrate that a DNA double strand was broken by Auger electrons generated on DNA, destructing spheroids.

When the results shown in FIG. 7 and FIG. 8 are analyzed in combination, it is considered that a DNA double strand is broken by irradiation with a monochromatic X-ray and the DNA breakage finally induces destruction of spheroids.

As mentioned above, the effect of X-ray irradiation on spheroid destruction was significantly observed at 33.2 keV as the energy of the K-edge of an iodine atom but not observed at a slightly lower energy level of 33.0 keV. Furthermore, it was found that the effect is observed at a slightly higher energy level of 33.4 keV but lower than 33.2 keV. More specifically, it was demonstrated that the effect of an iodine atom is the largest when a monochromatic X-ray having an energy around that of the K-edge.

### Test Example 6

### Safety of Iodine Atom-Containing Hoechst Compound

A Hoechst compound (normal compound containing no iodine atom) is generally considered to have low toxicity. The following experiment was carried out to confirm that iodine atom-containing Hoechst compounds have low toxicity. Iodine atom-containing Hoechst compounds (I-Hoechst, 3HCl) different in addition amount were each incubated with ovarian cancer OVCAR8 cells. Cytotoxicity was investigated by a commercially available kit in accordance with LDH (lactate dehydrogenase) method.

FIG. 9 shows the results of the cytotoxicity test. As shown in the figure, the iodine atom-containing Hoechst compound did not show toxicity at a concentration of 20 μM or less.

### Test Example 7

### Destruction of Hypoxic Spheroid

The effect of destroying spheroids was investigated in hypoxic conditions by the following test.

The iodine atom-containing Hoechst compound (I-Hoechst) and spheroids were incubated (24 hours) in the same manner as in Test Example 3 to obtain spheroids having the Hoechst compound taken up therein. The spheroids were put in tubes and further incubated in a hypoxic (0% oxygen concentration) condition for further 18 hours. As a control, spheroids were incubated in a normal (not hypoxic) oxygen condition (20% oxygen concentration) in the same manner for 18 hours. After incubation in a hypoxic (or normoxic) condition, the spheroids were irradiated with monochromatic X-rays in the same manner as in Test Example 5. In the Test, whether spheroids are placed in a hypoxic condition was checked by inducing expression of a hypoxic marker, HIF-1α. Note that, HIF-1α is a kind of a hypoxia inducible factor and measurable by a commercially available kit.

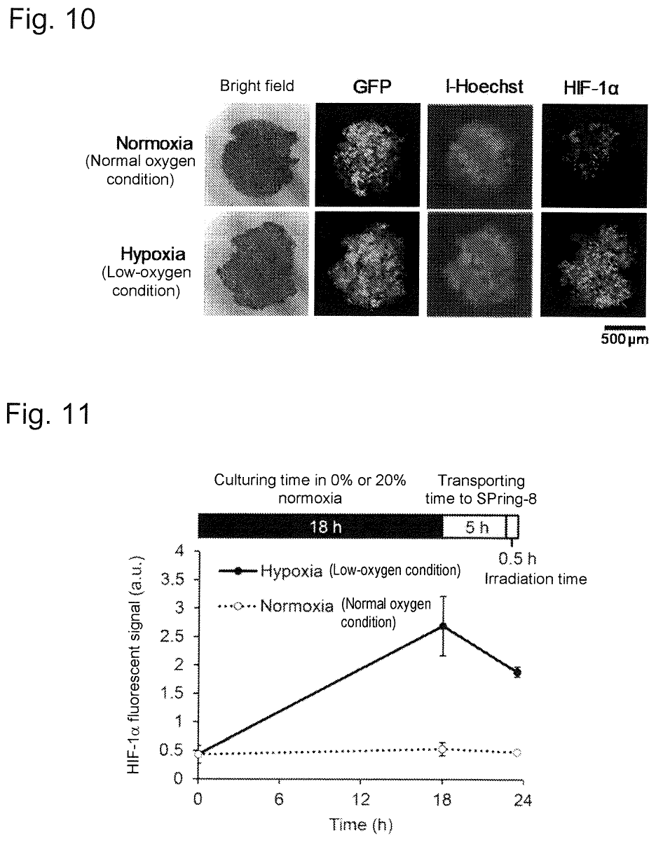

FIG. 10 shows the results (images) of microscopic observation on spheroids after incubation in a hypoxic condition and a normoxic condition. Furthermore, FIG. 11 shows a graph indicating changes over time of the fluorescence signal intensity of HIF-1α. As shown in these figures, it was confirmed that HIF-1α was expressed by incubation in the hypoxic condition although it is not expressed in the normoxic condition, indicating the formation of hypoxic spheroids. Note that, as shown in the figure, although there is a time lag (5 hours) until actual irradiation time due to transportation to an X radiation irradiation device, it was confirmed that hypoxic condition is maintained before irradiation.

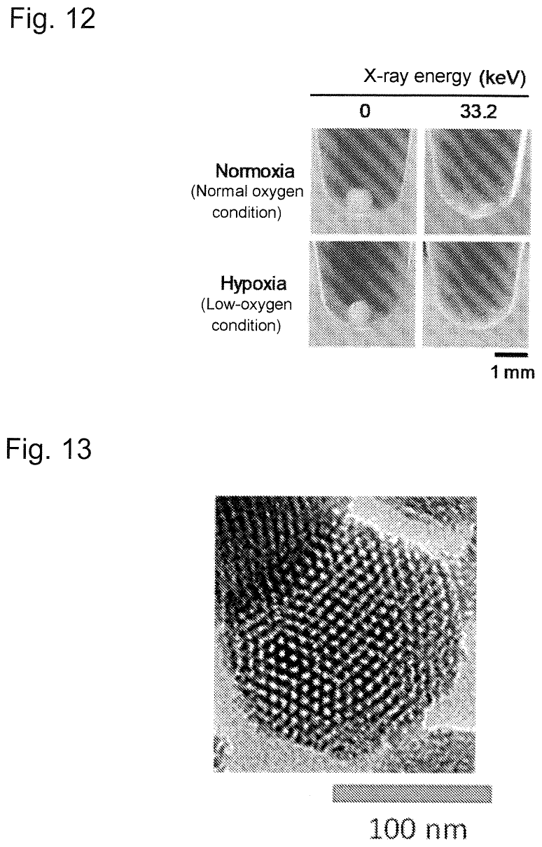

FIG. 12 shows the results of spheroids (images at the bottom of a tube) irradiated with X-rays. As shown in the figure, not only spheroids irradiated in a normoxia condition but also spheroids irradiated in a hypoxic condition were effectively destroyed. Accordingly, it was suggested that cancer treatment can be effectively performed by the method of the present invention even in a hypoxic condition in which a common radiotherapy cannot sufficiently produce an effect.

### Production Example 1

### Production of Nanoparticles Carrying an Iodine Atom-Containing Hoechst Compound

### Synthesis of Biodegradable Mesoporous Silica Nanoparticles

As a porous silica carrier, biodegradable mesoporous silica nanoparticles (BPMO) were synthesized by the following method.

A mixture of cetyltrimethylammonium bromide (CTAB, 98%, Sigma-Aldrich) (250 mg), an 8 M NaOH aqueous solution (219 μL) and water (120 mL) were vigorously stirred at 80° C. to prepare a CTAB solution. Separately, rhodamine B isothiocyanate (RITC, Sigma-Aldrich) (2.5 mg) was dissolved in ethanol (5 mL) and 3-aminopropyltriethoxysilane (APTS, 99%, Wako) (6 μL) was added thereto. The obtained mixture was stirred at room temperature for 30 minutes to prepare an RITC-APTS solution. Thereafter, 1,2-bis(triethoxysilyl)ethane (300 μL, Fluorochem) was mixed with the RITC-APTS solution. The obtained mixed solution was added dropwise in the CTAB solution. Immediately after that, bis[3-(triethoxysilyl)propyl]tetrasulfide (100 μL, Fluorochem) was added. The mixture was stirred for 15 minutes. Then, to this mixture, a monosodium 3-(trihydroxysilyl)propylmethylphosphonate aqueous solution (50%) (315 μL) was added and stirred. A solid product was obtained, collected by centrifugation and washed twice with ethanol. The solid product was subjected to reflux with a mixed solution of ammonium nitrate (0.3 g) and ethanol (50 mL). In this manner, CTAB serving as a template was removed. Thereafter, the solid product was centrifuged, washed three times with ethanol and dried overnight to obtain biodegradable mesoporous silica nanoparticles (BPMO).

FIG. 13 shows a TEM (transmission electron microscope) image of a mesoporous silica nanoparticle. A mesoporous is found in this image.

Loading of Iodine Atom-Containing Hoechst Compound into Nanoparticles

The iodine atom-containing Hoechst compound, “Hoechst 33342 analog 2 trihydrochloride” described above was dissolved in water to prepare a 0.8 mg/mL solution. The solution (110 μL) was added in an Eppendorf tube containing 1 mg of silica nanoparticles (BPMO). The mixture was stirred and mixed by a rotary mixer (80 rpm) at 4° C. for 24 hours, and subjected to centrifugation (14,000 rpm, room temperature, 10 minutes). After the supernatant was removed, the nanoparticles were washed three times with 110 μL of water to obtain nanoparticles carrying the iodine atom-containing Hoechst compound.

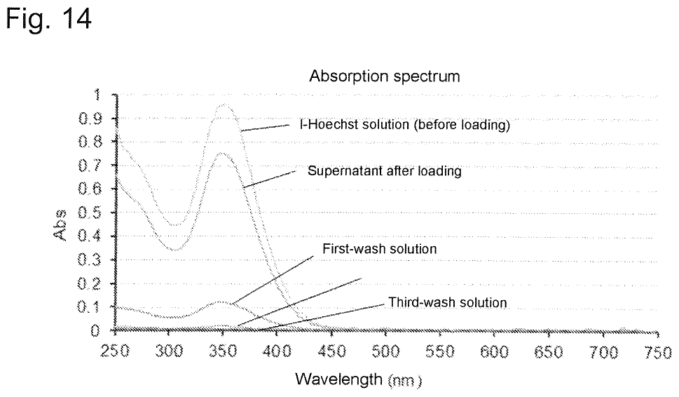

FIG. 14 shows the absorption spectra of solutions and washing solutions used for producing nanoparticles, measured by a spectrophotometer. The figure shows the absorption spectra of a solution of the iodine atom-containing Hoechst compound (before loading), the supernatant after loading, a first-wash solution, a second-wash solution and a third-wash solution. Absorbance was measured by an ultramicrospectrophotometer, NanoDrop.

Furthermore, Table 3 shows absorbance values at a wavelength of 346 nm and the NanoDrop dilution ratios (in the NanoDrop method, samples were diluted).

TABLE 3

| | Absorbance | NanoDrop |
|---|---|---|
| | (346 nm) | dilution ratio |
| Solution of the iodine atom- | 0.951 | 4 |
| containing Hoechst compound | | |
| Supernatant after loading | 0.753 | 4 |
| First-wash solution | 0.121 | 4 |
| Second-wash solution | 0.019 | 4 |
| Third-wash solution | 0.004 | 4 |

As shown above, as the number of washing times increases, absorption by the Hoechst compound decreases and adsorption at the third wash is virtually zero. Accordingly, it is demonstrated that the Hoechst compound is strongly carried by silica nanoparticles.

Furthermore, in this experiment, it was confirmed that when 1 mg of silica nanoparticles and 0.088 mg of the iodine atom-containing Hoechst compound were reacted, about 0.005 mg of the iodine atom-containing Hoechst compound were loaded.

### Test Example 8

### Uptake of Nanoparticles Carrying Iodine Atom-Containing Hoechst Compound by Cancer Cell

A test of uptake by cancer cells is performed in the same manner as in Test Example 2 except that nanoparticles carrying an iodine atom-containing Hoechst compound (the same amount in terms of the Hoechst compound) are used in place of the iodine atom-containing Hoechst compound. From the test, it is confirmed that the nanoparticles carrying an iodine atom-containing Hoechst compound are efficiently taken up by cancer cells.

Furthermore, a test of uptake by spheroids is performed in the same manner as in Test Example 3 except that nanoparticles carrying an iodine atom-containing Hoechst compound (the same amount in terms of the Hoechst compound) are used in place of the iodine atom-containing Hoechst compound. From the test, it is demonstrated that the nanoparticles are uniformly distributed within a spheroid and the permeability of nanoparticles into the spheroid is excellent.

Furthermore, the safety of the nanoparticles carrying an iodine atom-containing Hoechst compound is confirmed in the same manner as in Test Example 6.

### Test Example 9

### Irradiation of Monochromatic X-Rays to Nanoparticles Carrying an Iodine Atom-Containing Hoechst Compound-Spheroid

Spheroids (nanoparticles carrying an iodine atom-containing Hoechst compound-spheroid) prepared in Test Example 8 are irradiated with monochromatic X-rays in the same manner as in Test Example 5. After irradiation with the X-rays, the destruction of spheroids is confirmed. More specifically, it is confirmed that spheroids are completely destructed by irradiation of a 33.2-keV X-ray.

Furthermore, spheroids (nanoparticles carrying an iodine atom-containing Hoechst compound-spheroid) prepared in Test Example 8 are irradiated with monochromatic X-rays under a hypoxic condition in the same manner as in Test Example 7. It is confirmed that hypoxic spheroids irradiated with the X-rays are destroyed. More specifically, it is confirmed that hypoxic spheroids irradiated with a 33.2-keV X-ray are completely destroyed.

### Test Example 10

Nanoparticles (IH-BPMO) carrying an iodine atom-containing Hoechst (I-Hoechst) were added to a medium containing cancer cells and culture was performed for 24 hours in accordance with Test Example 8. Fluorescence was observed by a microscope. The results are shown in FIG. 15. Fluorescence from I-Hoechst was observed in nuclei. As a result of delivering IH by IH-BPMO, nuclear staining was confirmed.

### Test Example 11

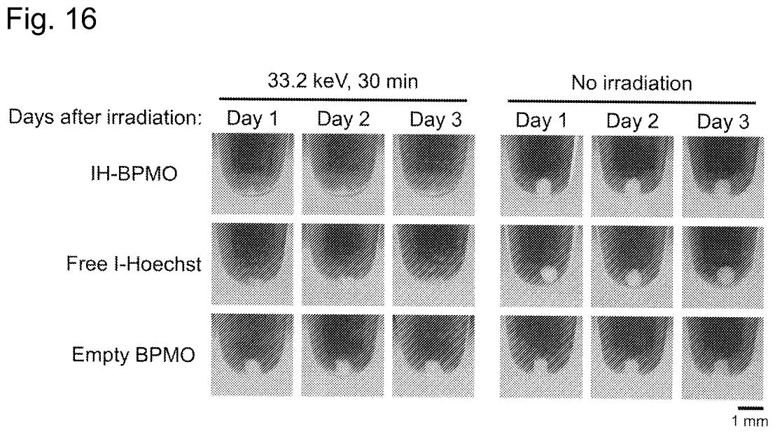

Nanoparticles (IH-BPMO) carrying an iodine atom-containing Hoechst (I-Hoechst) were added to a medium containing cancer spheroids and incubation was performed in accordance with Test Example 9 and then irradiated with monochromatic X-rays. The results are shown in FIG. 16. The destruction of cancer spheroids was confirmed.

### INDUSTRIAL APPLICABILITY

A highly effective radiotherapy can be performed by a pharmaceutical composition according to the present invention for radiotherapy.

1. A pharmaceutical composition comprising: a compound represented by formula (I): 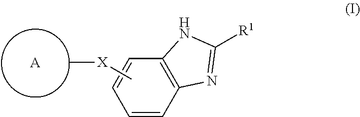
wherein
R¹ is an aryl group substituted with at least one substituent selected from the group consisting of an iodine atom, a C₁-C₄ alkyl group substituted with at least one iodine atom, an aryl group substituted with at least one iodine atom, a gadolinium atom-containing group, a gold atom-containing group, a silver atom-containing group and a platinum atom-containing group, wherein the aryl group is optionally substituted with at least one substituent selected from the group consisting of a hydroxy group, a C₁-C₄ alkyl group and a C₁-C₄ alkoxy group,
X represents a bond or a C₂ or C₄ alkenylene group, and
ring A is a group represented by formula (A): 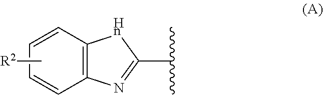
wherein
R² is a non-aromatic heterocyclic group containing one or more heteroatoms selected from the group consisting of a nitrogen atom, an oxygen atom and a sulfur atom, wherein at least one of the heteroatoms is a nitrogen atom, the non-aromatic heterocyclic group optionally substituted with at least one C₁-C₄ alkyl group, and [symbol]
represents the attachment to the group X,
or a pharmaceutically acceptable salt thereof; and a pharmaceutically acceptable carrier.

2. The pharmaceutical composition according to claim 1, wherein R¹ is an aryl group substituted with at least one iodine atom, wherein the aryl group is optionally substituted with at least one substituent selected from the group consisting of a hydroxy group, a C₁-C₄ alkyl group and a C₁-C₄ alkoxy group, and
R² is a 6-membered non-aromatic heterocyclic group containing 1 to 4 nitrogen atoms, optionally substituted with at least one C₁-C₄ alkyl group.

3. The pharmaceutical composition according to claim 1, wherein the compound represented by formula (I) is a compound represented by formula (II): 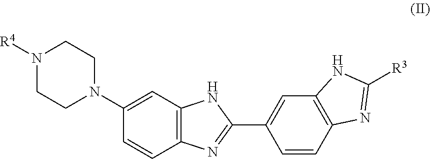
wherein
R³ is a phenyl group substituted with 1 to 3 iodine atoms, wherein the phenyl group is optionally substituted with 1 or 2 hydroxy groups, and
R⁴ is a hydrogen atom or a C₁-C₄ alkyl group.

4. (canceled)

5. (canceled)

6. (canceled)

7. A method for treating solid cancer or suppressing enlargement or growth of solid cancer, comprising irradiating, with an X-ray, the pharmaceutical composition according to claim 1, taken in a body of a subject, to destroy a cancer cell.

8. The method according to claim 7, wherein the solid cancer is a brain tumor, lung cancer, ovarian cancer, digestive system cancer, osteosarcoma or head and neck cancer.

9. The method according to claim 7, wherein the solid cancer is in a hypoxic condition.

10. A nanoparticle comprising:
a compound represented by formula (I): 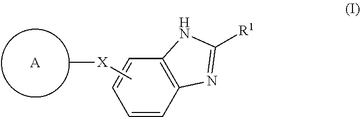
wherein
R¹ is an aryl group substituted with at least one substituent selected from the group consisting of an iodine atom, a C₁-C₄ alkyl group substituted with at least one iodine atom, an aryl group substituted with at least one iodine atom, a gadolinium atom-containing group, a gold atom-containing group, a silver atom-containing group and a platinum atom-containing group, wherein the aryl group is optionally substituted with at least one substituent selected from the group consisting of a hydroxy group, a C₁-C₄ alkyl group and a C₁-C₄ alkoxy group,
X represents a bond or a C₂ or C₄ alkenylene group, and
ring A is a group represented by formula (A): 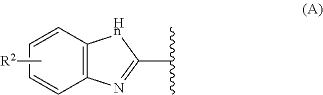
wherein
R² is a non-aromatic heterocyclic group containing one or more heteroatoms selected from the group consisting of a nitrogen atom, an oxygen atom and a sulfur atom, wherein at least one of the heteroatoms is a nitrogen atom, the non-aromatic heterocyclic group optionally substituted with at least one C₁-C₄ alkyl group, and [symbol]
represents the attachment to the group X,
or a pharmaceutically acceptable salt thereof; and
a porous silica carrier.

11. The nanoparticle according to claim 10, wherein the nanoparticle is a biodegradable mesoporous silica nanoparticle.

12. (canceled)

13. A method for treating solid cancer or suppressing enlargement or growth of solid cancer, comprising irradiating, with an X-ray, the compound represented by formula (I) or a pharmaceutically acceptable salt thereof according to claim 1, taken in a body of a subject, to destroy a cancer cell.

14. A method for treating solid cancer or suppressing enlargement or growth of solid cancer, comprising irradiating, with an X-ray, the nanoparticle according to claim 10, taken in a body of a subject, to destroy a cancer cell.

15. The method according to claim 13, wherein the solid cancer is a brain tumor, lung cancer, ovarian cancer, digestive system cancer, osteosarcoma or head and neck cancer.

16. The method according to claim 13, wherein the solid cancer is in a hypoxic condition.

17. The method according to claim 14, wherein the solid cancer is a brain tumor, lung cancer, ovarian cancer, digestive system cancer, osteosarcoma or head and neck cancer.

18. The method according to claim 14, wherein the solid cancer is in a hypoxic condition.

19. The method of claim 7, wherein the X-ray is an X-ray that can excite K shell electrons of an atom selected from an iodine atom, a gadolinium atom, a gold atom, a silver atom and a platinum atom of the compound represented by formula (I).

20. The method of claim 7, wherein the X-ray is a monochromatic X-ray or a characteristic X-ray.

21. The method of claim 13, wherein the X-ray is a monochromatic X-ray or a characteristic X-ray.

22. The method of claim 14, wherein the X-ray is an X-ray that can excite K shell electrons of an atom selected from an iodine atom, a gadolinium atom, a gold atom, a silver atom and a platinum atom of the compound represented by formula (I).

23. The method of claim 14, wherein the X-ray is a monochromatic X-ray or a characteristic X-ray.

24. The nanoparticle according to claim 10, wherein the nanoparticle is a degradable mesoporous silica nanoparticle.
